(19) United States
(12) Patent Application Publication
(10) Pub. No.: US 2026/0125418 A1
(43) Pub. Date: May 7, 2026

(54) A COMPOUND AND ITS USE FOR DETECTING A DISEASE IN THE EXHALED BREATH OF A SUBJECT

(71) Applicant: OWLSTONE MEDICAL LIMITED, Cambridge (GB)

(72) Inventors: Bryan WITTMANN, Cambridge (GB); Matthew HART, Cambridge (GB); Max ALLSWORTH, Cambridge (GB); Marc VAN DER SCHEE, Cambridge (GB); Christiaan LABUSCHAGNE, Cambridge (GB)

(21) Appl. No.: 19/117,957

(22) PCT Filed: Oct. 3, 2023
(86) PCT No.: PCT/GB2023/052544

§ 371 Date: Apr. 2, 2025

(30) Foreign Application Priority Data
Oct. 3, 2022 (GB) 2214528.8

(51) Int. Cl. C07K 1/107; C07B 59/00; G01N 33/58; G01N 33/84

(52) U.S. Cl. CPC C07K 1/1077; C07B 59/008; G01N 33/58; G01N 33/84; G01N 2800/085

(57) ABSTRACT

The invention relates to a compound of the formula Xₘ—(Y-L-T)ₙ and a method for the detection or prognosis of a disease in the exhaled breath of a subject. X, when present is a moiety operable to target the compound to a site of interest in vivo; m is (or at least 1, Y is a cleavable group; L is a self-immolative linker, T is a terminating moiety, and n is 1 when m is 0 or n is at least 1 when m is at least 1, L is a self-immolative linker. The terminating moiety, T, may comprise an isotopically labelled reporter molecule or a precursor thereof. The reporter molecule is operable to be released upon cleavage of the cleavable group, Y; and the reporter molecule, upon release, is a volatile compound.

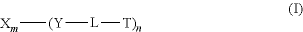

### FIELD OF THE INVENTION

The invention relates to compounds, compositions and kits that may be used in methods for the detection of prognosis of a disease.

### INTRODUCTION

Early diagnosis of cancer remains an important goal in any treatment plan. Cancer that is diagnosed at an early stage is more likely to be treated successfully. If the cancer spreads, effective treatment becomes more difficult, and generally a person's chances of surviving are much lower.

Examples of the benefits of early detection apply to lung cancer, breast cancer, ovarian cancer and bowel cancer. For example, more than 80% of lung cancer patients will survive for at least a year if diagnosed at the earliest stage compared to around 15% for people diagnosed with the most advanced stage of disease. As a further example, more than 90% of women diagnosed with breast cancer at the earliest stage survive their disease for at least 5 years compared to around 15% for women diagnosed with the most advanced stage of disease.

Whilst early detection of cancer is paramount, there are few non-invasive test methods available which enable a reliable diagnosis whilst increasing patient compliance due to the convenient testing methods. Moreover, there is also a need for monitoring the progression of cancer in a reliable and non-invasive way to determine treatment options.

Tumor cells are characterized by metabolic changes during the earliest stages of their development. Measuring the biochemicals related to these metabolic changes can therefore provide diagnostic biomarkers with a potential utility for the early detection of cancer (Muthu & Nordstrom, 2019). These biomarkers can be detected in bodily fluids such as breath, urine and blood. An attractive matrix for detection of these metabolites is breath as it can be accessed fully non-invasively at point of care, therefore lowering the threshold for participation in screening (Hakim et al., 2012).

Recently, the Exogenous Volatile Organic Compound (EVOC) Probe approach has been pioneered which enables active investigation of disease specific pathways. In this approach exogenous metabolic probe compounds are administered to patients which are metabolized by disease specific pathways resulting in a volatile product released in the patient breath (Gaude et al., 2018). There is a need to develop EVOC probes that can be used during non-invasive point of care. There is also a need for reliable non-invasive screening methods for early detection of cancer.

### SUMMARY OF THE INVENTION

In a first aspect, the invention relates to a compound of the formula Xₘ—(Y-L-T)ₙ, wherein X, when present, is an antigen-biding moiety, a ligand for a cell-surface receptor and/or a pH low insertion peptide (pHLIP); m is 0 or at least 1; Y is a cleavable group; L is a self-immolative linker; T is a terminating moiety; and n is 1 when m is 0 or n is at least 1 when m is at least 1; wherein at least one of the self-immolative linker, L, and terminating moiety, T, comprise an isotopically labelled reporter molecule or a precursor thereof; wherein the reporter molecule is operable to be released upon cleavage of the cleavable group, Y; and wherein the reporter molecule, upon release, is a volatile compound.

In a second aspect, the invention relates to a composition comprising the compound according to the first aspect of the invention.

In a third aspect, the invention relates to a method for the detection or prognosis of a disease comprising administering a compound according to the first aspect of the invention or a composition according to the second aspect of the invention to a subject and detecting the presence or absence of the released reporter molecule in the exhaled breath of the subject.

In a fourth aspect, the invention relates to a kit comprising the compound according to the first aspect of the invention and/or the composition according to the second aspect of the invention and a device for capturing a breath sample from a patient.

In a fifth aspect, the invention relates to the use of a compound according to the first aspect of the invention, a composition according to the second aspect of the invention and/or the kit according to the fourth aspect of the invention in a method according to third aspect of the invention.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. Suitable assays to measure the properties of the molecules disclosed herein are also described in the examples.

### X

X is a moiety operable to target the compound to a site of interest in vivo.

By “operable to target the compound to a site of interest”, and like terms as used herein, is suitably meant that X increases the specificity of the compound to a site of interest such that the compound is caused to accumulate at said site of interest.

X is suitably operable to target (or localise) the compound of the invention to a site of interest upon administration of the compound to a subject. The site of interest may be any suitable site. For example, the site of interest may be a tumour, a cell, tissue and/or organ, such as the liver. By way of example only, the site of interest may be a tumour when a subject has, or is suspected of having, a cancer. By way of example only, the site of interest may be a liver cell (or tissue), when a subject has, or is suspected of having, a liver disease.

Typically, X may target the site of interest by accumulating at said site. For example, X may target the site of interest by binding to a moiety that is associated with the site of interest, for example, by binding to a protease that is upregulated at the site of interest, anchoring into the membrane of a cell of interest, and/or biding to an antigen present on the cell surface of the site of interest.

X may comprise an antigen-binding moiety, a ligand for a cell surface receptor, a pHLIP, and/or a nanoparticle.

m is 0 or at least 1. n is 1 when m is 0. n is at least 1 when m is at least 1. It will therefore be appreciated that X is optional.

In some embodiments, m may be 0. In such embodiments, n is 1 and the compound is suitably of the formula Y-L-T, wherein each of Y, L and T are as defined herein.

In some embodiments, m may be 1. In such embodiments, the compound is suitably of the formula X—(Y-L-T)ₙ, wherein each of Y, L, T and n are as defined herein. When n is 2, the compound is suitably of the formula:

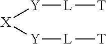

wherein each of X, Y, L and T are as defined herein. It will be appreciated by a person skilled in the art that in such embodiments when n is 3, 4, 5, 6 etc., the compound of the invention will suitably have 3, 4, 5, 6, etc., —(Y-L-T) groups attached to X.

In some embodiments, m may be 2 and n may be 1. In such embodiments, the compound is suitably of the formula:

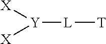

wherein each of Y, L and T are as defined herein. m may be 3 or more, for example, 3, 4, 5, 6, etc., and n may be 1. It will be appreciated by a person skilled in the art that when m is 3, 4, 5, 6 etc., and n is 1, the compound of the invention will suitably have 3, 4, 5, 6, etc., X groups attached to the cleavable group, Y.

For the avoidance of doubt, when X is at least 2 and n is at least 2, the compound may have any suitable structure on the proviso that each X group is attached to at least one Y group.

When m is at least 2, each X group may be the same or different. Preferably, when each X group may be different. The use of different X groups may advantageously enhance the ability to target the compound to a site of interest (i.e., by utilising two or more targeting methods, such as those defined herein).

### Antigen-Binding Moiety

In some embodiments, X may be an antigen-binding moiety.

X may be any suitable antigen-binding moiety.

The antigen-binding moiety may be capable of binding to one or more antigen(s) expressed by cells, such as by liver cells or cancer cells. As such, the antigen-binding moiety suitably binds to one or more antigen(s) expressed by cells, such as by liver cells or cancer cells.

The terms “antigen(s)” and “epitope(s)” are well established in the art and refer to the portion of a protein or polypeptide which is specifically recognized by a component of the immune system, e.g., an antibody or a T-cell/B-cell antigen receptor. As used herein, the term “antigen(s)” encompasses antigenic epitopes, e.g., fragments of antigens which are recognized by, and bind to, immune components. Epitopes can be recognized by antibodies in solution, e.g. free from other molecules. Epitopes can also be recognized by T-cell antigen receptors when the epitope is associated with a class I or class II major histocompatibility complex molecule.

The term “epitope” or “antigenic determinant” refers to a site on the surface of an antigen to which an immunoglobulin, antibody or antigen-binding fragment thereof specifically binds. Generally, an antigen has several or many different epitopes and reacts with many different antibodies. The term “specifically” includes linear epitopes and conformational epitopes.

Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antigen-binding fragment thereof (i.e., epitope mapping) are well known in the art and include, for example, immunoblotting and immunoprecipitation assays, wherein overlapping or contiguous peptides from are tested for reactivity with a given antibody or antigen-binding fragment thereof. Competition assays can also be used to determine if a test antibody binds to the same epitope as a reference antibody. Suitable competition assays are mentioned elsewhere herein and also shown in the examples. In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping).

The antigen-binding moiety may comprise an antibody.

The term “antibody” as used herein refers to an immunoglobulin (Ig) protein that is capable of binding an antigen. In particular, the term “antibody” as used herein broadly refers to any polypeptide comprising complementarity determining regions (CDRs) that confer specific binding affinity of the polypeptide for an antigen. The term antibody as used herein encompasses polyclonal and monoclonal antibody preparations.

As antibodies can be modified in a number of ways, the term “antibody” should be construed as covering antibody fragments, derivatives, functional equivalents and homologues of antibodies, including any polypeptide comprising an immunoglobulin binding domain. The term “antibody” should also be construed as covering antibody mimetics, such as, but not limited to, cyclic peptides, for example bicyclic peptides, cysteine knots and anticalins etc.

As such, the antigen-binding moiety may comprise an antibody and/or an antibody mimetic.

The antigen-binding moiety may comprise an antibody mimetic selected from a cyclic peptide, a cysteine knot and/or an anticalin.

The antigen-binding moiety may comprise a cyclic peptide, for example a bicyclic peptide.

The antigen-binding moiety may comprise a cysteine knot.

The antigen-binding moiety may comprise an anticalin.

The term “monoclonal antibody” refers to an antibody obtained from a single close of cells or cell line. The individual antibodies are identical and/or bind the same epitope. Unlike polyclonal antibodies, which include different antibodies directed against different epitopes, each monoclonal antibody of in a preparation is directed against a single epitope.

The antibody, or antigen-binding fragment thereof, described herein, “which binds” or is “capable of binding” the antigen of interest, binds the antigen with sufficient affinity such that the antibody or antigen-binding fragment thereof is useful as a therapeutic or diagnostic agent. The term “specific” may refer to the situation in which the antibody molecule will not show any significant binding to molecules other than its specific binding partner.

The terms “polypeptide(s)” and “protein(s)” are used interchangeably throughout the application and denote at least two covalently attached amino acids, thus may signify proteins, polypeptides, oligopeptides, peptides, and fragments thereof. The protein may be made up of naturally occurring amino acids and peptide bonds, or synthetic peptidomimetic structures. Hence, “amino acid(s)” or “peptide residue(s)”, as used herein, denote both naturally occurring and synthetic amino acids. In some cases, the immunoglobulin proteins of the present invention may be synthesized using any in vivo or in vitro protein synthesis technique known in the art.

In a full-length antibody, each heavy chain is comprised of a heavy chain variable region or domain (abbreviated herein as HCVR, VH or V_{H}) and a heavy chain constant region. The heavy chain constant region is comprised of three domains, CH₁, CH₂ and CH₃. Each light chain is comprised of a light chain variable region or domain (abbreviated herein as LCVR, VL or V_{L}) and a light chain constant region. The light chain constant region is comprised of one domain, C_{L}.

Antibodies may include the kappa (κ) and lambda (A) light chains and the alpha (IgA), gamma (IgG1, IgG2, IgG3, IgG4), delta (IgD), epsilon (IgE) and mu (IgM) heavy chains, or their equivalents in other species. Full-length immunoglobulin “light chains” (usually of about 25 kDa or 214 amino acids long) consist of a variable region of approximately 110 amino acids at the NH2-terminus and a kappa or lambda constant region at the COOH-terminus. Full-length immunoglobulin “heavy chains” (usually of about 50 kDa or 446 amino acids long), likewise consist of a variable region (of about 116 amino acids) and one of the aforementioned heavy chain constant regions, e.g. gamma (of about 330 amino acids).

Light or heavy chain variable regions are generally composed of a “framework” region (FR) interrupted by three hypervariable regions, also called CDRs. The extent of the framework region and CDRs have been precisely defined. The sequences of the framework regions of different light and heavy chains are relatively conserved within a species. The framework region of an antibody, i.e. the combined framework regions of the constituent light and heavy chains, serves to position and align the CDRs. The CDRs are primarily responsible for binding to an epitope of an antigen. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

Heavy chain CDRs are designated HCDR1, HCDR2 and HCDR3. Light chain CDRs are designated LCDR1, LCDR2 and LCDR3.

The antibody may be comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art.

Immunoglobulin molecules can be of any type (e.g., IgG, IgE, IgM, IgD, IgA and IgY), class (e.g., IgG1, IgG2, IgG3, IgG4, IgA1 and IgA2) or subclass. The antibody may be of the IgG type.

Different definitions of the CDRs are commonly in use. The method described by Kabat is the most commonly used and CDRs are based on sequence variability (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk, J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme (Lefranc et al., Dev. Comp. Immunol., 29, 185-203 (2005)). According to the IMGT numbering scheme, a CDR is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG).

The IMGT system as described above is used herein, unless otherwise stated. The terms “IMGT numbering”, “IMGT definitions” and “IMGT labelling” are used interchangeably herein. antigen-binding

The term “antibody” is not only inclusive of antibodies generated by methods comprising immunisation, but also includes any polypeptide, e.g., a recombinantly expressed polypeptide, which is made to encompass at least one CDR capable of specifically binding to an epitope on an antigen of interest. Hence, the term applies to such molecules regardless whether they are produced in vitro, in cell culture, or in vivo. Methods of producing polyclonal and monoclonal antibodies are known in the art and described more fully below.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant regions. Alternatively, a hybridoma or other cell producing an antibody molecule may be subject to genetic mutation or other changes, which may or may not alter the binding specificity of antibodies produced.

The term antibody as used herein is meant to specifically include antibody fragments/antigen-binding fragments thereof unless stated otherwise. The antibody fragment/antigen-binding fragments may be selected from any fragment capable of binding the antigen or antigenic fragment of interest. Exemplary antibody fragments include, but are not limited to, Fab, Fab′, F(ab′)2, F(ab′)3, Fabc, Fd, single chain Fv (scFv), (scFv)2, Fv, scFv-Fc, heavy chain only antibody, diabody, tetrabody, triabody, minibody, antibody mimetic protein, single domain antibody, e.g. a VH. Thus, the antibody fragment/antigen-binding fragment may comprise or consist of any of these fragments.

Antigen-binding fragments derived from an antibody, including single-chain antibodies, may comprise the variable region(s) alone or in combination with the entire, or parts of the, following: a heavy chain constant domain, or a portion thereof, e.g. a CH1, CH2, CH3, transmembrane, and/or cytoplasmic domain, on the heavy chain, and a light chain constant domain, e.g. a Ckappa or Clambda domain, or portion thereof on the light chain. Also included in the present disclosure are any combinations of variable region(s) and CH1, CH2, CH3, Ckappa, Clambda, transmembrane and cytoplasmic domains.

Fv fragments (˜25 kDa) consist of the two variable domains, VH and VL. Naturally, VH and VL domain are non-covalently associated via hydrophobic interaction and tend to dissociate. However, stable fragments can be engineered by linking the domains with a hydrophilic flexible linker to create a single chain Fv (scFv).

The smallest antigen-binding fragment is the single variable fragment, namely the variable heavy (VH) or variable light (VL) chain domain. VH and VL domains respectively are capable of binding to an antigen. They are generally referred to as a “single domain antibody” or “immunoglobulin single variable domain”. A single domain antibody (˜12 to 15 kDa) has thus either the VH or VL domain. Antigen-binding single VH domains have also been identified from, for example, a library of murine VH genes amplified from genomic DNA from the spleens of immunized mice and expressed in E. coli (Ward et al., 1989, Nature 341:544-546). Ward et al. named the isolated single VH domains “dAbs,” for “domain antibodies.” The term “dAb” or “sdAb” (for single domain antibody) generally refers to a single immunoglobulin variable domain (VH, VHH or VL) polypeptide that specifically binds antigen. For use in therapy, human single domain antibodies are preferred over camelid derived VHH, primarily because they are not as likely to provoke an immune response when administered to a patient.

The antibody or antigen-binding fragment thereof may be chimeric, human or humanised. A “chimeric antibody” is a recombinant protein that contains the variable domains including the CDRs of an antibody derived from one species, for example a murine antibody, while the constant domains of the antibody molecule are derived from those of a different species, for example a human antibody.

A humanised antibody is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. Methods to humanise antibodies include CDR grafting based on framework regions homology and antibody resurfacing. Human or humanised antibodies or antigen-binding fragments are most desirable for use in antibody diagnostics or therapies, as such molecules would elicit little or no immune response in the human subject.

The antigen-binding moiety may comprise a monoclonal antibody.

The antibody may comprise a CH2 domain. The CH2 domain is for example located at the N-terminus of the CH3 domain, as in the case in a human IgG molecule. The CH2 domain of the antibody may be the CH2 domain of human IgG1, IgG2, IgG3, or IgG4, e.g., the CH2 domain of human IgG1. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. The hinge region, or part thereof, may be a human IgG1, IgG2, IgG3 or IgG4 hinge region, or part thereof. For example, the hinge region, or part thereof, may be an IgG1 hinge region, or part thereof.

The sequence of the CH3 domain is not particularly limited. The CH3 domain may be a human immunoglobulin G domain, such as a human IgG1, IgG2, IgG3, or IgG4 CH3 domain, e.g. a human IgG1 CH3 domain.

The antibody may comprise a human IgG1, IgG2, IgG3, or IgG4 constant region. The sequences of human IgG1, IgG2, IgG3, or IgG4 CH3 domains are known in the art. The antibody may comprise a non-human IgG constant region, e.g., a rabbit IgG1 constant region.

The antibody may comprise a human IgG Fc with effector function.

The antibody may be modified to increase half-life, for example by a chemical modification, especially by PEGylation, or by incorporation in a liposome, or using a serum albumin protein or an antibody or antibody fragment that binds human serum albumin. Increased half-life can also be conferred by conjugating the molecule to an antibody fragment. The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms.

Half-life may be increased by at least 1.5 times, preferably at least 2 times, such as at least 5 times, for example at least 10 times or more than 20 times, greater than the half-life of the corresponding antibodies without such modification. For example, increased half-life may be more than 1 hours, preferably more than 2 hours, more preferably more than 6 hours, such as more than 12 hours, or even more than 24, 48 or 72 hours, compared to the corresponding antibodies without such modification. The in vivo half-life of the antigen-binding moiety or compound of the invention a can be determined in any manner known per se, such as by pharmacokinetic analysis. Suitable techniques will be clear to the person skilled in the art. Half-life can for example be expressed using parameters such as the t½-alpha t½-beta and the area under the curve (AUC). It will be appreciated by a person skilled in the art that reference to the half-life of an antibody may also refer to the half-life of the compounds of the invention (and may be used interchangeably herein).

The antibody may be produced by any suitable method. The antibody may be produced in/by murine, mammal or other animal models, by using hybridoma technology or other methods known in the art.

There are several methods by which to produce recombinant antibodies which are known in the art. One of these is production in an E. coli expression system. In the E. coli expression system, nucleic acids encoding the antibody or antigen-binding fragment thereof or binding molecule as described herein may be inserted into a plasmid and expressed in a suitable expression system. For example, the present invention includes methods for expressing an antibody or antigen-binding fragment thereof or immunoglobulin chain thereof in a host cell (e.g., bacterial host cell such as E. coli, CHO or other host cell) comprising expressing T7 RNA polymerase in the cell which also includes a polynucleotide encoding an immunoglobulin chain that is operably linked to a T7 promoter. For example, a bacterial host cell, such as an E. coli, may include a polynucleotide encoding the T7 RNA polymerase gene operably linked to a lac promoter and expression of the polymerase and the chain is induced by incubation of the host cell with IPTG (isopropyl-beta-D-thiogalactopyranoside).

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art.

Thus, the antibody may be produced by a method comprising the steps of (i) introducing one or more polynucleotides encoding light and/or heavy immunoglobulin domains of the antibody wherein the polynucleotide is in a vector; and/or integrated into a host cell chromosome and/or is operably linked to a promoter; (ii) culturing the host cell (e.g., E. coli, CHO or Pichia or Pichia pastoris) under conditions favourable to expression of the polynucleotide and, (iii) optionally, isolating the antibody from the host cell and/or medium in which the host cell is grown. When making an antibody comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antibody. The methods include those wherein only a heavy immunoglobulin chain or only a light immunoglobulin chain (e.g., any of those discussed herein including mature fragments and/or variable domains thereof) is expressed. Such chains are useful, for example, as intermediates in the expression of an antibody that includes such a chain.

The antigen-binding moiety may be a full length antibody. The full length antibody may comprise human constant regions and human light chain regions.

A skilled person will know that there are different ways to identify, obtain and optimise antibodies that may be used herein, including in vitro and in vivo expression libraries. Optimisation techniques known in the art, such as display (e.g., ribosome and/or phage display) and/or mutagenesis (e.g., error-prone mutagenesis) can be used. The invention therefore also may comprise sequence optimised antibody variants.

The antibody may bind to prostate specific membrane antigen (PSMA). PMSA is a prostate tumour associated marker. PSMA is a 750-residue type II transmembrane glycoprotein highly restricted to prostate secretory epithelial cell membranes. It is highly expressed in prostate cancer cells and in nonprostatic solid tumor neovasculature and expressed at lower levels in other tissues, including healthy prostate, kidney, liver, small intestine, and brain. PSMA expression increases with prostate disease progression and metastasis and its expression level has thus been correlated with tumour aggressiveness. Various immunohistological studies have demonstrated increased PSMA levels in virtually all cases of prostatic carcinoma compared to those levels in benign prostate epithelial cells. Intense PSMA staining is found in all stages of the disease, including prostatic intraepithelial neoplasia, late stage androgen-independent prostate cancer and secondary prostate tumours localized to lymph nodes, bone, soft tissue, and lungs. PSMA is thus widely used as a biomarker for prostate cancer cells.

PSMA has a 3-part structure: a 19-amino-acid internal portion, a 24-amino-acid transmembrane portion, and a 707-amino-acid external portion. It forms a noncovalent homodimer that possesses glutamate carboxypeptidase activity based on its ability to process the neuropeptide N-acetylaspartylglutamate and glutamate-conjugated folate derivatives. PSMA is rapidly and efficiently internalized by an endocytic pathway and rapidly recycles back to the membrane.

The antibody may comprise a first human single heavy chain variable immunoglobulin (VH) domain antibody capable of binding specifically to human PSMA, optionally comprising a second human single heavy chain variable immunoglobulin (VH) domain antibody capable of binding specifically to human PSMA and optionally comprising a third human single heavy chain variable immunoglobulin (VH) domain antibody.

Suitable antibodies that bind to human PSMA are disclosed in WO 2017/122019, which is fully incorporated herein by reference.

As defined herein, the antigen-binding moiety may comprise an antibody mimetic. The antibody mimetic may be any suitable antibody mimetic. Examples of suitable antibody mimetics include, but are not limited to, cyclic peptides, such as bicyclic peptides, cysteine knots, anticalin and combinations thereof.

The antigen-binding moiety may comprise a cyclic peptide, preferably a bicyclic peptide.

The antigen-binding moiety may comprise a cyclic peptide specific for nectin cell adhesion molecule 4 (NECTIN4), ephrin receptor A2 (EPHA2) and/or a membrane type 1 metalloprotease (MT1-MMP), such as membrane type 1 metalloprotease 14 (MT1-MMP14). The term “specific for” in this context refers to a peptide that is operable to bind one or more of the aforementioned, or other, moieties.

The antigen-binding moiety may comprise a cyclic peptide specific for a membrane type 1 metalloprotease (MT1-MMP), such as membrane type 1 metalloprotease 14 (MT1-MMP14), The terms “specific for MT1-MMP” and “specific for MT1-MMP14” as used herein refers to peptides that are operable to bind to an MT1-MMP and/or MT1-MMP14. MT1-MMP is a transmembrane metalloprotease that plays a major role in the extracellular matrix remodelling, directly by degrading several of its components and indirectly by activating pro-MMP2. MT1-MMP is crucial for tumour angiogenesis (Sounni et al (2002) FASEB J. 16 (6), 555-564) and is over-expressed on a variety of solid tumours, such as non-small cell lung carcinomas.

The cyclic peptide may be specific for human MT1-MMP, such as human MT1-MMP14.

The cyclic peptide specific for MT1-MMP typically comprises a peptide covalently bound to a molecular scaffold. Typically, such peptides comprise two or more reactive groups (i.e., cysteine residues) which are capable of forming covalent bonds to the scaffold, and a sequence subtended between said reactive groups which is referred to as the loop sequence, since it forms a loop when the peptide is bound to the scaffold. The peptide may comprise at least three cysteine residues and form at least two loops on the scaffold.

The cyclic peptide, which may suitably be specific for MT1-MMP, may comprise a peptide comprising at least three cysteine residues, separated by at least two loop sequences, and a molecular scaffold which forms covalent bonds with the cysteine residues of the polypeptide such that at least two polypeptide loops are formed on the molecular scaffold, wherein the peptide ligand comprises an amino acid sequence of formula (I):

formula (I)
(SEQ ID NO: 1)
-C-X₁-U/O-X₂-X₃-G-C-E-D-F-Y-X₄-X₅-C-

or a pharmaceutically acceptable salt thereof; wherein each of X₁, X₂, X₃, X₄ and X₅ independently represents any amino acid residue; U represents a polar, uncharged amino acid residue selected from the group consisting of N, C, Q, M, S and T; and O represents a non-polar aliphatic amino acid residue selected from the group consisting of G, A, I, L, P and V.

By “pharmaceutically acceptable salt,” and like terms as used herein, is meant pharmaceutically acceptable organic or inorganic salts of a compound. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Thus, a pharmaceutically acceptable salt can have one or more charged atoms and/or one or more counterion.

As described herein, all bicyclic peptides are assumed to be cyclised with TBMB (1,3,5-tris(bromomethyl)benzene) yielding a tri-substituted 1,3,5-trismethylbenzene structure. Cyclisation with TBMB occurs on the cysteine residues.

X₁ may be selected from Y, M, F or V, for example Y, M or F, for example Y or M, or for example Y.

U/O may be a U, for example N. When U/O is a U, the peptide ligand may suitably comprise an amino acid sequence of the formula -C-X₁-U-X₂-X₃-G-C-E-D-F-Y-X₄-X₅-C-formula (I) (SEQ ID NO: 2).

U/O may be an O, for example G. When U/O is an O, the peptide ligand may suitably comprise an amino acid sequence of the formula -C-X₁-O-X₂-X₃-G-C-E-D-F-Y-X₄-X₅-C-formula (I) (SEQ ID NO: 3).

X₂ may be selected from U or Z, wherein U represents a polar, uncharged amino acid residue selected from N, C, Q, M, S and T, for example Q, and Z represents a polar, negatively charged amino acid residue selected from D or E, for example E.

X₃ may be selected from J, wherein J represents a non-polar aromatic amino acid residue selected from F, W and Y.

X₄ may be selected from Z, wherein Z represents a polar, negatively charged amino acid residue selected from D or E, for example D.

X₅ may be selected from O, wherein O represents a non-polar aliphatic amino acid residue selected from G, A, I, L, P and V, for example I.

The cyclic peptide may suitably be substantially pure. By “substantially pure” is meant that the cyclic peptide suitably has at least 90 to 95% homogeneity. The cyclic peptide may have at least 98%, or at least 99% homogeneity.

Further details of suitable cyclic peptides that are specific for a membrane type 1 metalloprotease (MT1-MMP), such as membrane type 1 metalloprotease 14 (MT1-MMP14), are disclosed in WO 2017/191460, which is fully incorporated herein by reference.

The antigen-binding moiety may bind to an antigen with any suitable binding affinity. “Binding affinity” generally refers to the strength of the sum total of non-covalent interactions between a single binding site of a molecule (e.g., an antibody or antibody-mimetic) and its binding partner (e.g., an antigen). Unless indicated otherwise, as used herein, “binding affinity” refers to intrinsic binding affinity which reflects a 1:1 interaction between members of a binding pair (e.g., antibody or antigen-mimetic and antigen). The affinity of a molecule X for its partner Y can generally be represented by the dissociation constant (KD).

Affinity can be measured and/or expressed in a number of ways known in the art, including, but not limited to, equilibrium dissociation constant (KD), and equilibrium association constant (KA). The KD is calculated from the quotient of koff/kon, whereas KA is calculated from the quotient of kon/koff. Kon refers to the association rate constant of, e.g., an antibody or antigen-mimetic to an antigen, and koff refers to the dissociation of, e.g., an antibody or antigen-mimetic from an antigen. The affinity can be determined by techniques known to one of ordinary skill in the art, such as surface plasmon resonance (SPR) or KinExA.

The antigen-binding moiety may have an affinity KD of <250 nM, <200 nM, <100 nM, <50 nM, <10 nM or <1 nM for the target antigen.

### Ligand for a Cell-Surface Receptor

In some embodiments, X may be a ligand for a cell-surface receptor.

The ligand for a cell-surface receptor may be any suitable ligand.

The ligand for a cell-surface receptor may be capable of binding to one or more receptors on the surface of a cell, such as a liver cell or cancer cell.

Examples of suitable ligands include, but are not limited to, folic acid, folate and/or peptide hormones.

Examples of suitable peptide hormones include, but are not limited to, somatostatins and/or somatostatin analogues.

It will be appreciated that when the ligand is folate, for example, the ligand may suitably bind to a folate receptor. Similarly, it will be appreciated that when the ligand is a somatostatin and/or a somatostatin analogue, the ligand may suitably bind to a somatostatin receptor (SSTR), for example to SSTR1, SSTR2, SSTR3, SSTR4 and/or SSTR5. Somatostatin receptor are G protein-coupled transmembrane receptors (GPCR).

### pH Low Insertion Peptide

In some embodiments, X may be a pH low insertion peptide (pHLIP®).

The pHLIP® may suitably be operable to anchor in a cell membrane, such as in the cell membrane of a liver cell or a tumour cell, for example. By the term “anchor in a cell membrane”, and like terms as used herein, is meant that the peptide may insert into the lipid bilayer of a cell membrane.

A pHLIP® is a water-soluble membrane peptide that interacts weakly with a cell membrane at neutral pH, without insertion into the lipid bilayer. However, at slightly acidic pH (<7.0), a pHLIP® inserts into the cell membrane and, on the proviso that it is long enough and non-cyclic, can form a stable transmembrane alpha-helix. Tumour cells, for example, are characterized by a low surface pH.

The pHLIP® may comprise a sequence selected from the group consisting of:
ALDDQNPWRAYLDLLFPTDTLLLDLLWA (SEQ ID NO: 4),
ACDDQNPWRAYLDLLFPTDTLLLDLLWA (SEQ ID NO: 5),
AX¹DDQNPWRAYLDLLFPTDTLLLDLLWA (SEQ ID NO: 6),
ALDQDNPWRAYLDLLFPTDTLLLDLLWA (SEQ ID NO: 7),
ACDQDNPWRAYLDLLFPTDTLLLDLLWA (SEQ ID NO: 8),
AX¹DQDNPWRAYLDLLFPTDTLLLDLLWA (SEQ ID NO: 9), AX² (X³) nX²PWRAYLDLLFPTDTLLLDLLWA (SEQ ID NOS: 10-19, wherein n=1 for SEQ ID NO: 10, n=2 for SEQ ID NO: 11, n=3 for SEQ ID NO: 12 etc., up to n=10 for SEQ ID NO: 19), and wherein X¹ represents an azido-containing amino acid; X² represents any amino acid residue, such as a lysine (Lys), a cysteine (Cys) or an azido-containing amino acid; X³ represents any amino acid, and n represents an integer from 1 to 10. For example, (X³) n may be QDNDQN such that the pHLIP® is of the sequence AX²QDNDQNX²PWRAYLDLLFPTDTLLLDLLWA (SEQ ID NO: 20) or any combination of polar amino acid residues including D, E, N and/or Q.

Further examples of suitable pHLIP® sequences are disclosed in WO 2020/160047, which is hereby incorporated by reference in full. In particular, examples if suitable pHLIP® sequences are provided in Table 1, Table 2, Table 6 and Table 7 of WO 2020/160047. The pHLIP® may be commercially available from pHLIP, Inc.

### Nanoparticle

In some embodiments, X may be a nanoparticle.

The nanoparticle may suitably be operable to preferentially accumulate the compound at a site of interest (as defined herein). For example, nanoparticle may have enhanced permeability and/or retention at the site of interest.

Examples of suitable nanoparticles include, but are not limited to, iron oxide, gold nanoparticles, non-metallic nanoparticles and combinations thereof. Gold nanoparticles typically consist of a core of gold atoms that may be functionalized by addition of a monolayer of moieties containing a thiol (SH) group.

Advantageously, due to tumour tissue properties and the size and surface characteristics of nanoparticles, the use of nanoparticles can increase permeability and retention of the compound at the tumour tissue. This may result in accumulation of the compound at the tumour site.

Nanoparticles may suitably have an average particle size from 1 to 1000 nm, such as from 10 to 100 nm.

The nanoparticle may comprise a protein nanoparticle. Protein nanoparticles may be generated using proteins, such as fibroins, albumin, gelatin, gliadine, legumin, lipoprotein, and ferritin proteins, and are typically prepared through emulsion, electrospray and/or desolvation methods (Hong S, Choi D W, Kim H N, Park C G, Lee W, Park H H. Protein-Based Nanoparticles as Drug Delivery Systems. Pharmaceutics. 2020; 12 (7): 604. Published 2020 Jun. 29). For example, albumin, a plasma protein with a molecular weight of 66 kDa, has been extensively investigated as a drug carrier.

The nanoparticle may comprise a synthetic chemical polymer nanoparticle. Polymeric nanoparticles have been extensively investigated as drug nanocarriers. The most widely researched synthetic polymers include polylactide (PLA), poly(D,L-lactide-co-glycolide) (PLGA) and PEG. All three polymers are hydrolysable in vivo and are biodegradable (Malam Y, Loizidou M, Seifalian A M. Liposomes and nanoparticles: nanosized vehicles for drug delivery in cancer. Trends Pharmacol Sci. 2009 November; 30 (II): 592-9).

Preferably, the nanoparticle may comprise a gold nanoparticle and/or an albumin nanoparticle.

### Cleavable Group, Y

Y is a cleavable group.

The cleavable group, Y, may be cleaved by any suitable means. For example, the cleavable group may be enzymatically cleaved and/or may be hydrolytically cleaved. The cleavable group, Y, may be caused to undergo hydrolytic cleavage by one or more of the following conditions: altered pH; reducing conditions; hypoxic conditions; the action of reactive oxygen species; and combinations thereof.

Y may be any suitable cleavable group.

The cleavable group, Y, may comprise a peptide and/or a glucuronide group.

In some embodiments, the cleavable group, Y, may comprise a peptide.

The peptide is suitably operable to be enzymatically cleaved, such as by one or more protease(s).

The peptide may be designed and optimized for selectivity for enzymatic cleavage by a particular protease, for example, by a tumour-associated protease or a protease associated with liver disease (including those as defined herein, for example).

The peptide may be a dipeptide, an oligopeptide or a polypeptide. The peptide may comprise from 2 to 20 amino acids, such as from 2 to 12 amino acids, such as from 2 to 10 amino acids, such as 2, 3, 4, 5, 6, 7 or 8 amino acids.

The peptide may be a dipeptide.

The peptide may be of formula -(—C(O)—CH₂(R⁴)—NR⁵—)_{w}-, wherein w is an integer from 2 to 12; each R⁴ is independently hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄ NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

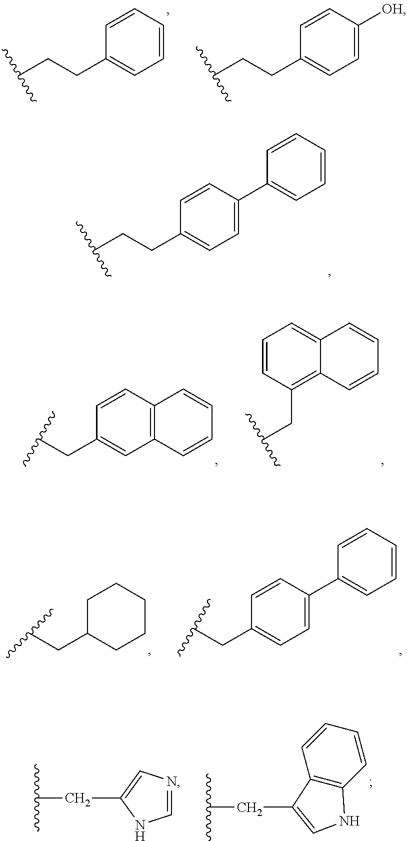

and each R⁵ is independently H or CH₃.

Each amino acid of the peptide may be natural or unnatural. Each amino acid of the peptide may be a D- or L-isomer.

Each amino acid of the peptide may independently be selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid and/or derivatives of each of the aforementioned amino acids.

Each amino acid of the peptide linker, P, may independently be an L-(natural) amino acid, such as alanine, arginine, aspartic acid, asparagine, histidine, glycine, glutamic acid, glutamine, phenylalanine, lysine, leucine, serine, tyrosine, threonine, isoleucine, tryptophan and/or valine.

Each amino acid of the peptide linker may independently be a D-isomer of the following natural amino acids: alanine, arginine, aspartic acid, asparagine, histidine, glycine, glutamic acid, glutamine, phenylalanine, lysine, leucine, serine, tyrosine, threonine, isoleucine, tryptophan and valine.

Examples of alanine and derivatives thereof include but are not limited to, alanine (Ala), N-alkyl-alanine, dehydro-alanine, 4-thiazolylalanine, 2-pyridylalanine, 3-pyridylalanine, 4-pyridylalanine, β-(1-naphthyl)-alanine, β-(2-naphthyl)-alanine, α-aminobutyric acid, β-chloro-alanine, β-cyano-alanine, β-cyclopentyl-alanine, β-cyclohexyl-alanine, β-iodo-alanine, β-cyclopentenyl-alanine, β-tBu-alanine, β-cyclopropyl-alanine, β-diphenyl-alanine, β-fluoro-alanine, β-piperazinyl-alanine with the piperazine ring protected or not, β-(2-quinolyl)-alanine, β-(1,2,4-triazol-I-yi)-alanine, β-ureido-alanine, H-β-(3-benzothienyl)-Ala-OH, and H-β-(2-thienyl)-Ala-OH.

Examples of arginine and derivatives thereof include but are not limited to, arginine (Arg), N-alkyl-arginine, H-Arg(Me)-OH, H-Arg(NH₂)—OH, H-Arg(NO₂)—OH, H-ATg(Ac)₂—OH, H-Arg(Me)₂-OH (asymmetrical), H-Arg(Me)₂-OH (symmetrical), 2-amino-4-(2′-hydroxyguanidino)-butyric acid (N-w-hydroxy-nor-arginine) and homoarginine.

Examples of aspartic acid and derivatives thereof include, but are not limited to, aspartic acid (Asp), N-alkyl-aspartic acid, and H-Asp (OtBu)-OH.

Examples of asparagine and derivatives thereof include, but are not limited to, asparagine (Asn), N-alkyl-asparagine, and isoasparagine (H-Asp-NH₂).

Examples of cysteine and derivatives thereof, such as those containing no free SH group, include, but are not limited to, H-Cys(Acm)-OH, H-Cys(Trt)-OH, H-Cys(tBu)-OH, H-Cys(Bzl)-OH, H-Cys(Et)-OH, H-Cys(SO₃H)—OH, H-Cys(aminoethyl)-OH, H-Cys(carbamoyl)-OH, H-Cys(phenyl)-OH, H-Cys(Boc)-OH, and H-Cys(hydroxyethyl)-OH.

Examples of histidine and derivatives thereof include, but are not limited to, histidine (His), N-alkyl-histidine, H-His (Boc)-OH, H-His (Bzl)-OH, H-His (1-Me)-OH, H-His (1-Tos)-OH, H-2,5-diiodo-His-OH, and H-His (3-Me)-OH.

Examples of glycine and derivatives thereof include, but are not limited to, glycine (Gly), N-alkyl-glycine, H-propargylglycine, α-aminoglycine (protected or not), β-cyclopropyl-glycine, cyclopentyl-glycine, cyclohexyl-glycine, α-allylglycine, t-butyl-glycine, neopentylglycine and phenylglycine.

Examples of glutamic acid and derivatives thereof include, but are not limited to, glutamic acid (Glu), N-alkyl-glutamic acid, H-Glu (OtBu)-OH, H-γ-hydroxy-Glu-OH, H-γ-methylene-Glu-OH, H-γ-carboxy-Glu (OtBu)₂-OH, and pyroglutamic acid.

Examples of glutamine and derivatives thereof include, but are not limited to, glutamine (Gln), N-alkyl-glutamine, isoglutamine (H-Glu-NH₂), H-Gln(Trt)-OH, and H-Gln(isopropyl)-OH.

Examples of phenylalanine and derivatives thereof include, but are not limited to, phenylalanine (Phe), N-alkyl-phenylalanine, H-p-amino-Phe-OH, H-p-amino-Phe(Z)-OH, H-p-bromo-Phe-OH, H-p-Benzyl-Phe-OH, H-p-tBu-Phe-OH, H-p-carboxy-Phe (OtBu)-OH, H-p-carboxy-Phe-OH, H-p-cyano-Phe-OH, H-p-fluoro-Phe-OH, H-3,4-dichloro-Phe-OH, H-p-iodo-Phe-OH, H-p-nitro-Phe-OH, H-p-methyl-Phe-OH, H-pentafluoro-Phe-OH, H-m-fluoro-Phe-OH, H-α-Me-Phe-OH, H-4-phenyl-Phe-OH, homophenylalanine, chloro-phenylalanine and β-homophenylalanine.

Examples of lysine and derivatives thereof include, but are not limited to, lysine (Lys), N-alkyl-lysine, H-Lys(Boc)-OH, H-Lys(Ac)—OH, H-Lys(Formyl)-OH, H-Lys(Me)₂-OH, H-Lys(nicotinoyl)-OH, H-Lys(Me) 3-OH, H-trans-4,5-dehydro-Lys-OH, H-Lys(Aloc)-OH, H—H-δ-hydroxy-Lys-OH, H-δ-hydroxy-Lys(Boc)-OH, H-Lys(acetamidoyl)-OH, and H-Lys(isopropyl)-OH

Examples of leucine and derivatives thereof include, but are not limited to, leucine (Leu), N-alkyl-leucine, 4,5-dehydroleucine, H-α-Me-Leu-OH, homoleucine, norleucine, and t-leucine.

Examples of methionine and derivatives thereof include, but are not limited to, methionine (Met), H-Met(O)—OH, and H-Met(O)₂—OH.

Examples of serine and derivatives thereof include, but are not limited to, serine (Ser), N-alkyl-serine, H-Ser(Ac)—OH, H-Ser(tBu)-OH, H-Ser(Bzl)-OH, H-Ser(p-chloro-Bzl)-OH, H-β-(3,4-dihydroxyphenyl)-Ser-OH, H-β-(2-thienyl)-Ser-OHJ isoserine N-alkyl-isoserine, and 3-phenyliso serine.

Examples of tyrosine and derivatives thereof include, but are not limited to, tyrosine (Tyr), N-alkyl-tyrosine, H-3,5-dinitro-Tyr-OH, H-3-amino-Tyr-OH, H-3,5-dibromo-Tyr-OH, H-3,5-diiodo-Tyr-OH, H-Tyr (Me)-OH, H-Tyr (tBu)-OH, H-Tyr (Boc)-OH, H-Tyr (Bzl)-OH, H-Tyr (Et)-OH, H-3-iodo-Tyr-OH, and H-3-nitro-Tyr-OH.

Examples of threonine and derivatives thereof include, but are not limited to, threonine (Thr), N-alkyl-threonine, allo-threonine, H-Thr(Ac)—OH, H-Thr(tBu)-OH, and H-Thr(Bzl)-OH.

Examples of isoleucine and derivatives thereof include, but are not limited to, isoleucine (Ile), N-alkyl-isoleucine, allo-isoleucine, and norleucine.

Examples of tryptophan and derivatives thereof include, but are not limited to, tryptophan (Trp), N-alkyl-tryptophan, H-5-Me-Trp-OH, H-5-hydroxy-Trp-OH, H-4-Me-Trp-OH, H-α-Me-Trp-OH, H-Trp(Boc)-OH, H-Trp(Formyl)-OH, and H-Trp(Mesitylene-2-sulfonyl)-OH.

Examples of proline and derivatives thereof include, but are not limited to, proline (Pro), N-alkyl-proline, homoproline, thioproline, hydroxyproline (H-Hyp-OH), H-Hyp(tBu)-OH, H-Hyp(Bzl)-OH, H-3,4-dehydro-Pro-OH, 4-keto-proline, α-Me-Pro-OH, and H-4-fluoro-Pro-OH.

Examples of valine and derivatives thereof include, but are not limited to, valine (Val), N-alkyl-valine, H-α-Me-Val-OH, and norvaline.

Examples of ornithine and derivatives thereof include but are not limited to: ornithine, N-alkyl-ornithine, H-Orn(Boc)-OH, H-Orn(Z)-OH, H-α-difluoro-Me-Orn-OH (Eflornitine), and H-Orn(Aloc)-OH.

Examples of penicillamine and derivatives thereof include, but are not limited to, penicillamine, H-penicillamme(Acm)-OH (H-β,β-dimethylcys(Acm)-OH) and N-alkyl-penicillamine.

Examples of β-alanine and derivatives thereof include, but are not limited to, β-alanine, N-alkyl-β-alanine, and dehydro-alanine.

Examples of an aminoalkanoic acid and derivatives thereof include, but are not limited to, N-alkylaminoalkanoic acid, aminobutyric acid, 4-(neopentyloxysulfonyl)-aminobutyric acid, ε-aminocaproic acid, α-aminoisobutyric acid, piperidylacetic acid, 3-amrnopropionic acid, 3-amino-3-(3-pyridyl)-propionic acid, and 5-aminopentanioic acid (amino valeric acid).

Examples of an aminoalkynoic acid and derivatives thereof include, but are not limited to, N-alkylaminoalkynoic acid, 6-amino-4-hexynoic acid, and 6-(Boc-amino)-4-hexynoic acid.

Examples of an aminoalkanedioic acid and derivatives thereof include, but are not limited to, N-alkylaminoalkanedioic acid, 2-aminohexanedioic acid, 2-aminoheptanedioic acid and 2-aminooctanedioic acid (H-Asu-OH).

Examples of an aminobenzoic acid and derivatives thereof include, but are not limited to, N-alkylaminobenzoic acid, 2-aminobenzoic acid, 3-aminobenzoic acid, and 4-aminobenzoic acid.

Examples of an amino-heterocyclo-alkanoic acid and derivatives thereof include, but are not limited to, N-alkylamino-heterocyclo-alkanoic acids, 4-amino-1-methyl-1H-imidazol-2-carboxylic acid, 4-amino-1-methyl-1H-pyrrole-2-carboxylic acid, 4-amino-piperidine-4-carboxylic acid (H-Pip-OH; 1-protected or not), and 3-amino-3-(3-pyridyl)-propionic acid.

Examples of a heterocyclo-carboxylic acid and derivatives thereof include, but are not limited to, azetidine-2-carboxylic acid, azetidine-3-carboxylic acid, piperidine-4-carboxylic acid, and thiazolidine-4-carboxylic acid.

Examples of citrulline and derivatives thereof include, but are not limited to, citrulline (cit), N-alkyl-citrulline, thio citrulline, S-methyl-thiocitrulline, and homocitrulline.

Examples of statine and derivatives thereof include, but are not limited to, statine, N-alkyl-statine, cyclohexylstatine, and phenylstatine.

Examples of diaminoalkanoic acid (Dab) and derivatives thereof include, but are not limited to, N-alkyl-diamino-alkanoic acids, N,N-dialkylamino-alkanoic acids, α,γ-diaminobutyric acid (H-Dab-OH), H-Dab(Aloc)-OH, H-Dab(Boc)-OH, H-Dab(Z)-OH, α,β-diaminopropionic acid and its side-chain protected versions.

The peptide may comprise any suitable combination of amino acids. For example, the peptide may comprise only natural amino acids, may comprise only non-natural amino acids or may comprise a combination of natural and non-natural amino acids. The peptide linker, P, may comprise a natural amino acid and a D-isomer of a natural amino acid.

At least one amino acid of the peptide may be an L-amino acid. At least one amino acid of the peptide may be a D-amino acid. At least one amino acid of the peptide may have a chiral centre in the S-configuration. At least one amino acid of the peptide may have a chiral centre in the R-configuration.

The peptide may be selected from the group consisting of: Phe-Lys, Val-Lys, Phe-Phe-Lys, (D) Phe-Phe-Lys, Gly-Phe-Lys, Ala-Lys, Val-Ala, Val-Cit, Phe-Cit, Leu-Cit, Ile-Cit, Trp-Cit, Phe-Ala, Gly-Phe-Leu-Gly, Ala-Leu-Ala-Leu, Phe-N⁹-tosyl-Arg, Phe-N⁹-nitro-Arg, (L) Lys-Met, (L) Lys-Asn, (D) Asp-Tyr, (D) Asp-Nva, (D) Lys-Phg, (D) Lys-Met, and -(D) Lys-Asn.

The peptide may be selected from the group consisting of: Phe-Lys, Val-Lys, Phe-Phe-Lys, D-Phe-Phe-Lys, Gly-Phe-Lys, Ala-Lys, Val-Ala, Val-Cit, Phe-Cit, Leu-Cit, Ile-Cit, Trp-Cit, Phe-Ala, Gly-Phe-Leu-Gly, Ala-Leu-Ala-Leu, Phe-N⁹-tosyl-Arg and Phe-N⁹-nitro-Arg.

The peptide may be selected from the group consisting of: Phe-Lys, Val-Lys, Val-Ala, Val-Cit and D-Phe-L-Phe-Lys.

The peptide may be selected from the group consisting of: Val-Ala and Val-Cit.

The peptide may be selected from the group consisting of: (L) Lys-Met and (L) Lys-Asn.

The peptide may be selected from the group consisting of: (D) Asp-Tyr, (D) Asp-Nva, (D) Lys-Phg, (D) Lys-Met, and -(D) Lys-Asn.

The peptide may not contain cysteine, proline and/or an N-methyl amino acid.

When m is 1 and the cleavable group, Y, is a peptide, the peptide may be covalently linked to X. When the peptide is covalently linked to X, the N-terminus of the peptide may be linked directly to X. Alternatively, the N-terminus of the peptide may be linked indirectly to X via an acyl unit. For example, if X has an amino-reactive group, then the acyl unit may not be necessary (although it may still be employed). However, if X does not have an amino-reactive group, then an acyl unit may conveniently be included. The acyl unit suitably contains an acyl group that may be reacted with the N-terminus of the peptide and also contains a second reactive group that is reactive with a functional group on X. Thus, an acyl unit may be defined as a bifunctional agent containing separate reactive sites, the first of which is a carboxylic acid or a reactive equivalent thereof. This first reactive site may be joined to the N-terminus of the peptide through an amide linkage. The second reactive site is suitably used to couple to X.

Examples of suitable second reactive sites include, but are not limited to, maleimides and haloacetamides that may be used to react with thiol groups on X; hydrazides that react with aldehydes and ketones on the on X; and hydroxysuccinimides, isocyanates, isothiocyanates, and anhydrides that react with amino groups on X.

The second reactive site may be a maleimide. For example, the peptide may be selected from the group consisting of: maleimidecaproyl-Val-Cit, maleimidecaproyl-Val-Ala, maleimidomethyl cyclohexane-1-carboxylate-Val-Cit and maleimidomethyl cyclohexane-1-carboxylate-Val-Ala.

Suitable bifunctional reactive linker groups are well known in the art (for example, S. S. Wong, Chemistry of Protein Conjugation and Cross-Linking, CRC Press, Inc., Boston, 1991).

The acyl unit may be of the following formula:

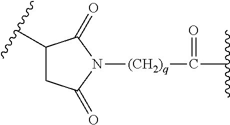

wherein q is an integer from 1 to 10, such as from 3 to 6, or even 5.

In some embodiments, the cleavable group, Y, may comprise a glucuronide group.

The glucuronide group suitably includes a site that can be cleaved by a β-glucuronidase enzyme.

The glucuronide group suitably comprises a sugar moiety, Su.

The sugar moiety, Su, may suitably be linked via a glycoside bond (-G′-) to the self-immolative linker, L. As such, the compound of the invention may be of formula Xₘ-(-[Su-G′]-L-T) wherein each of X, m, L and T are as defined herein; Su is a sugar moiety; and -G′-is a glycosidic bond. The glycosidic bond is typically a β-glucuronidase-cleavage site, such as a bond cleavable by human, lysosomal β-glucuronidase. As such, the glycosidic bond (-G′-) is suitably operable to be cleaved by a β-glucuronidase, such as lysosomal β-glucuronidase. Additionally or alternatively, the glycosidic bond may be cleaved by other means, such as by hydrolysis, for example.

As used herein the term “glycoside” has its usual meaning in the art and refers to a compound comprising a carbohydrate portion usually a sugar molecule or uronic acid molecule which is linked to a non-sugar molecule via a glycosidic bond. The sugar portion or uronic acid portion may be referred to as the “glycone” and the non-sugar portion may be referred to as the “aglycone”. Suitably, the glycone is a glycoside of glucuronic acid also referred to as glucuronide.

As mentioned above the glycoside comprises a glycosidic bond. The glycosidic bond links the glycone molecule and the aglycone molecule of the glycoside. There are multiple types of glycosidic bond that may be present, for example the glycosidic bond may be selected from an —O—, —N—, —S— or —C-glycosidic bond. The glycosidic bond is preferably an —O-glycosidic bond. Thus, the glucuronide unit suitably comprises a sugar moiety, Su, linked via a glycoside bond (—O′—) to the self-immolative linker, L. As such, the compound of the invention may be of formula Xₘ-(-[Su-O′]-L-T) wherein each of X, m, L and T are as defined herein; Su is a sugar moiety; and —O′— is a glycosidic bond. A glycosidic bond is formed between the hemiacetal or hemiketal group of the sugar or uronic acid molecule and the aglycone. The glycosidic bond may adopt an α or a β stereochemistry.

The sugar moiety, Su, may be a cyclic hexose, such as a pyranose, or a cyclic pentose, such as a furanose. The pyranose may be a glucuronide or hexose. The sugar moiety, Su, may suitably be in the β-D conformation. For example, the pyranose may be a β-D-glucuronide moiety (i.e., β-D-glucuronic acid linked to the self-immolative group, I, via a glycosidic bond that is cleavable by β-glucuronidase). The sugar moiety, Su, may be unsubstituted. For example, the sugar moiety, Su, may be a naturally occurring cyclic hexose or cyclic pentose. The sugar moiety, Su, may be a substituted β-D-glucuronide (i.e., glucuronic acid substituted with one or more group, such as hydrogen, hydroxyl, halogen, sulfur, nitrogen or lower alkyl).

The sugar moiety, Su, may be ethyl-BD-glucuronide.

The sugar moiety may be labelled or unlabelled. For example, the sugar moiety, Su, may comprise D5-ethyl-βD-glucuronide.

When the sugar moiety, Su, is ethyl-βD-glucuronide and/or D5-ethyl-βD-glucuronide, it will be appreciated that cleavage of the glycosidic bond, whether by enzymatic or other means, e.g., hydrolysis, for example, will suitably result in the release of ethanol and/or D5-ethanol.

Advantageously, use of a labelled sugar moiety, Su, provides a further means of detection. For example, the released D5-ethanol may be detected along with one or more of the reporter molecules described herein.

In some embodiments, the cleavable group, Y, may comprise two or more cleavable groups, Y. In such embodiments, the compound of the invention may suitably be of the formula Xₘ—(Yₙ-L-T), wherein each of X, m, Y, L and T are as defined herein and n is 2 to 5, for example 2 to 4, for example 2 or 3, for example 2. In such embodiments, each cleavable group, Y, may be the same or different. For example, in some embodiments, the cleavable group, Y, may comprise a peptide and a glucuronide group (such as the peptide and glucuronide groups defined herein). In such embodiments, the peptide and glucuronide groups may be present in any suitable configuration. For example, the compound may be of the formula Xₘ-(-[Su-G′]-P-L-T), wherein each of X, m, Su, G′, L and T are as defined herein; and P is a peptide. For example, the compound may be of the formula Xₘ-(—P-[Su-G′]-L-T), wherein each of X, m, Su, G′, L and T are as defined herein; and P is a peptide. Any other combination of peptide and glucuronide groups is within the scope of the invention.

In some embodiments, when m is 1, the cleavable group, Y, may be covalently linked directly to X. In such embodiments, the compound of the invention may be of the formula X—(Y-L-T), wherein each of X, Y, L and T are as defined herein.

In some embodiments, when m is 1, the cleavable group, Y, may comprise one or more stretcher unit(s), S, between X and the cleavable group, Y. In such embodiments, the compound of the invention may be of the formula X-(—Sₓ—Y-L-T), wherein each of X, Y, L and T are as defined herein; S is a stretcher unit; and x is 1 or 2.

The stretcher unit, S, is suitably capable of linking X to the cleavable group, Y. Suitably, therefore, X may have a functional group that can form a bond with a functional group of the stretcher unit, S. Examples of suitable functional groups that may be present on X either naturally or via chemical manipulation, include, but are not limited to, sulfhydryl (—SH), amino, hydroxyl, carboxy, the anomeric hydroxyl group of a carbohydrate, carboxyl and/or combinations thereof. X may comprise sulfhydryl and/or amino functional groups. Sulfhydryl groups can be generated by reduction of an intramolecular disulphide bond of X. Alternatively, sulfhydryl groups can be generated by reaction of an amino group of a lysine moiety of X using 2-iminothiolane (Traut's reagent) and/or another sulfhydryl generating reagent.

x may be 1 and the stretcher unit, S, may form a bond with the cleavable group, Y. x may be 2 and one of the stretcher units, S, may form a bond with the cleavable group, Y.

The stretcher unit, S, may form a bond with a sulphur atom of X.

The stretcher unit, S, may also be linked to X via a disulphide bond between a sulphur atom of X and a sulphur atom of the stretcher unit, S.

The stretcher unit, S, may contain a reactive site that can form a bond with a primary or secondary amino group of X. Examples of suitable reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates, isothiocyanates and/or combinations thereof.

The stretcher unit, S, may contain a reactive site that is reactive to a modified carbohydrate's (—CHO) group that may be present on X. For example, a carbohydrate can be mildly oxidized using a reagent such as sodium periodate and the resulting (—CHO) unit of the oxidized carbohydrate may be condensed with a stretcher unit, S, that contains a functionality such as a hydrazide, an oxime, a primary or secondary amine, a hydrazine, a thiosemicarbazone, a hydrazine carboxylate, and/or an arylhydrazide such as those described by Kaneko et al. (1991) Bioconjugate Chem 2:133-41.

The stretcher unit, S, may be —NH—R¹—R² or —O—R¹—R², wherein —R¹—R² has the formula:

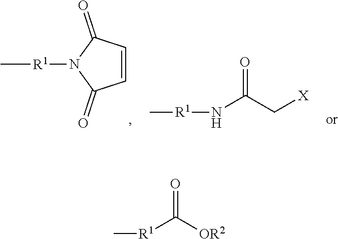

wherein R¹ may be selected from the group consisting of C₁-C₁₀ alkylene, C₃-C₈ cycloalkylene, arylene, C₁-C₃₀ heteroalkylene, C₃-C₈ heterocycloalkylene, C₁-C₁₀ alkylene-arylene, arylene-C₁-C₁₀ alkylene-, C₁-C₁₀ alkylene-(C₃-C₈ cycloalkylene), (C₃-C₈ cycloalkylene)-C₁-C₁₀ alkylene, C₁-C₁₀ alkylene-(C₃-C₈ heterocycloalkylene)-, and (C₃-C₈ heterocycloalkylene)-C₁-C₁₀ alkylene; wherein X is a leaving group; and each R² forms an activated ester, wherein R² is independently selected from the group consisting of H, C₁-C₁₀ alkyl, C₃-C₈ cycloalkyl, aryl, C₁-C₃₀ heteroalkyl, C₃-C₈ heterocycloalkylene, C₁-C₁₀ alkylene-aryl, arylene-C₁-C₁₀ alkyl, C₁-C₁₀ alkylene-(C₃-C₈ cycloalkyl), (C₃-C₈ cycloalkyl)-C₁-C₁₀ alkyl, C₁-C₁₀ alkylene-(C₃-C₈ heterocycloalkylene), and (C₃-C₈ heterocycloalkylene)-C₁-C₁₀ alkyl.

When the stretcher unit, S, is —NH—R¹—, —R¹— may be selected from —C₁-C₁₀ alkylene-, —C₁-C₁₀ alkylene-NH—C(O)—C₁-C₁₀ alkylene-, —C₁-C₁₀ alkylene-C(O)—NH—C₁-C₁₀ alkylene-, —(CH₂CH₂O)_{z}—, —CH₂CH₂O)_{z}—CH₂—, —(CH₂CH₂NH)_{z}—(CH₂)_{z}, —(CH₂CH₂NH)_{z}(CH₂)_{z}—NH—C(O)—(CH₂)_{z}, —(C₃-C₈ cycloalkyl)-, -arylene-, and —C₃-C₈ cycloalkyl-, wherein each z is independently 1-10.

When the stretcher unit, S, is —O—R¹—, —R¹— may be selected from —C₁-C₁₀ alkylene-, —C₁-C₁₀ alkylene-NH—C(O)—C₁-C₁₀ alkylene-, —C₁-C₁₀ alkylene-C(O)—NH—C₁-C₁₀ alkylene-, —(CH₂CH₂O)_{z}—, —(CH₂CH₂O)_{z}—CH₂—, —(C₃-C₈ cycloalkyl)-, -arylene-, and —C₃-C₈ heterocyclo-; wherein each z is independently 1-10.

When the stretcher unit, S, is —O—R¹-R¹⁰—, the ester may be a hindered ester.

x may be 1 or 2, for example 1.

The cleavable group, Y, suitably links the stretcher unit, S, to the self-immolative linker, L.

The cleavable group, Y, may be selectively stable, such that the cleavable group, Y, is not readily cleaved in situ, but is cleaved under conditions present in the microenvironment of a target cell.

The cleavable group, Y, may be labile through proteolysis, and may provide for a facile cleavage of the cleavable group, Y, under the action of one or more proteases that are present in the microenvironment of a target cell.

In some embodiments, the compound may comprise a further cleavable group, Y′. The further cleavable group, Y′, may be covalently attached to the compound at any suitable position. For example, the further cleavable group, Y′, may be attached to the self-immolative linker. L. In such embodiments, the compound may be of the formula: Xₘ—(Y-L(Y″)-T)ₙ.

The further cleavable group, Y′, may be any suitable group. The further cleavable group, Y′, may be a peptide and/or a glucuronide group (such as those peptide and glucuronide groups as defined herein).

In some embodiments, the cleavable group, Y, may be a peptide and the further cleavable group, Y′, may be a glucuronide group. In some embodiments, the cleavable group, Y, may be a glucuronide groups and the further cleavable group, Y′, may be a peptide group. In some embodiments, both the cleavable group, Y, and the further cleavable group, Y′, may be a peptide. In some embodiments, both the cleavable group, Y, and the further cleavable group, Y′, may be a glucuronide group.

When the compound comprises more than one peptide, each peptide may be the same or may be different, for example each peptide may be different. When the compound comprises more than one glucuronide group, each glucuronide group may be the same or may be different, for example each glucuronide group may be different.

Advantageously, the use of a further cleavable group, Y′, may improve the in vivo stability and/or tolerability of the compound.

### Self-Immolative Linker, L

L is a self-immolative linker.

By “self-immolative”, and like terms as used herein, is meant a chemical moiety, such as a bifunctional chemical moiety, which is capable of covalently linking together two spaced chemical moieties into a normally stable tripartite molecule, wherein the self-immolative chemical moiety will spontaneously separate from the second chemical moiety if its bond to the first chemical moiety is cleaved. The first and second chemical moieties may each be any suitable chemical moiety, such as those defined herein.

The self-immolative linker, L, may comprise a methylene carbamate group, an aminobenzyl carbamate group, an aminobenzyl carbonate group, an aminobenzyl ether group, a dithiobenzyl carbamate group, a dithiobenzyl carbonate group and/or a dithiobenzyl ether group.

The aminobenzyl carbamate group may be a p-aminobenzyl carbamate (PABC) group.

The aminobenzyl carbonate group maybe a p-aminobenzyl carbonate group.

The aminobenzyl ether group may be a p-aminobenzyl ether group.

The self-immolative linker, L, may comprise an aminobenzyl carbamate group. The aminobenzyl carbamate group may be of the following formula:

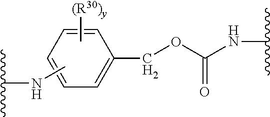

The self-immolative linker, L, may comprise an aminobenzyl carbonate group. The aminobenzyl carbonate group may be of the following formula:

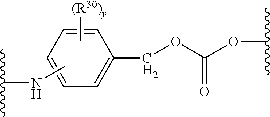

The self-immolative linker, L, may comprise an aminobenzyl ether group. The aminobenzyl ether group may be of the following formula:

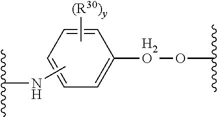

wherein, in each case the ‘bond’ from the amine functionality of the aminobenzyl group into the aromatic ring thereof indicates that the amine functionality may be bonded to any of the five carbon atoms that both form the ring and are not substituted with the methylene carbamate (—CH₂—O—C(O)—NH—), methylene carbonate (—CH₂—O—C(O)—O—) or methylene ether (—CH₂—O—) group that is necessarily bonded to the ring; y is an integer from 0 to 4; and R³⁰ is a substituent on the aromatic ring which, when present, replaces a hydrogen that is otherwise attached to one of the four non-substituted carbons that form the ring.

The amine functionality may be covalently bound to the aromatic ring at the para or ortho position on the ring relative to the methylene carbamate (—CH₂—O—C(O)—NH—), methylene carbonate (—CH₂—O—C(O)—O—) or methylene ether (—CH₂—O—) group. The amine functionality may be covalently bound to the aromatic ring at the para position on the ring relative to the methylene carbamate (—CH₂—O—C(O)—NH—), methylene carbonate (—CH₂—O—C(O)—O—) or methylene ether (—CH₂—O—) group. Thus, the aminobenzyl carbamate group may be a p-aminobenzyl carbamate (PABC) group. Thus, the aminobenzyl carbonate group may be a p-aminobenzyl carbonate group. Thus, the aminobenzyl ether group may be a p-aminobenzyl ether group.

The R³⁰ group, which may be a single atom, e.g., a halogen, or a multi-atom group, e.g., methyl, may be selected in order to impact the stability of the aminobenzyl carbamate or the decomposition product thereof. Electron withdrawal from the ring may be used to facilitate the spontaneous decomposition of the aminobenzyl group from the terminating moiety, T, after cleavage of the bond between the amino group of the aminobenzyl carbamate group and the adjacent peptide linkage. Exemplary R³⁰ substituents include, but are not limited to, F, Cl, Br, NO₂, NHCOCH₃, N(CH₃)₂, NHCOCF₃, alkyl, and/or haloalkyl.

y may be 0. It will be appreciated that when y is 0, there will be no substituents, R³⁰, present on the aromatic ring.

y may be 1. y may be 1 and R³⁰ may be an election deficient group, such as NO₂. y may be 1, R³⁰ may be an election deficient group, such as NO₂, and R³⁰ may be present at the meta position relative to the methylene carbamate (—CH₂—O—C(O)—NH—), methylene carbonate (—CH₂—O—C(O)—O—) or methylene ether (—CH₂—O—) group.

The self-immolative linker, L, may comprise a dithiobenzyl carbamate group. The dithiobenzyl carbamate group may be of the following formula:

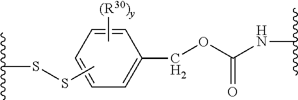

The self-immolative linker, L, may comprise a dithiobenzyl carbonate group. The dithiobenzyl carbonate group may be of the following formula:

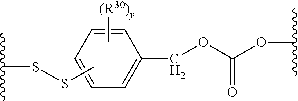

The self-immolative linker, L, may comprise a dithiobenzyl ether group. The dithiobenzyl ether group may be of the following formula:

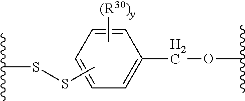

wherein, in each case, the ‘bond’ from the dithio functionality of the dithiobenzyl group into the aromatic ring thereof indicates that the dithio functionality may be bonded to any of the five carbon atoms that both form the ring and are not substituted with the methylene carbamate (—CH₂—O—C(O)—NH—), methylene carbonate (—CH₂—O—C(O)—O—) or methylene ether (—CH₂—O—) group that is necessarily bonded to the ring; y is an integer from 0 to 4; and R³⁰ is a substituent on the aromatic ring which, when present, replaces a hydrogen that is otherwise attached to one of the four non-substituted carbons that form the ring.

The dithio functionality may be covalently bound to the aromatic ring at the para or ortho position on the ring relative to the methylene carbamate (—CH₂—O—C(O)—NH—), methylene carbonate (—CH₂—O—C(O)—O—) or methylene ether (—CH₂—O—) group.

Each of the R³⁰ group and y are as defined herein in relation to the aminobenzyl carbamate, carbonate or ether groups.

In some embodiments, the self-immolative linker, L, may comprise a single self-immolative group (such as one of the methylene carbamate, aminobenzyl carbamate, aminobenzyl carbonate, aminobenzyl ether, dithiobenzyl carbamate, dithiobenzyl carbonate or dithiobenzyl ether groups as defined herein). In such embodiments, the compound of the invention may suitably be of the formula Xₘ—(Y-Lₙ-T), wherein each of X, m, Y, L and T are as defined herein and n is 1.

In some embodiments, the self-immolative linker, L, may comprise two or more self-immolative groups (such as two or more of the methylene carbamate, aminobenzyl carbamate, aminobenzyl carbonate, aminobenzyl ether, dithiobenzyl carbamate, dithiobenzyl carbonate and/or dithiobenzyl ether groups as defined herein). In such embodiments, the compound of the invention may suitably be of the formula Xₘ—(Y-Lₙ-T), wherein each of X, m, Y, L and T are as defined herein and n is 2 to 5, for example 2 to 4, for example 2 or 3, for example 2. In such embodiments, each self-immolative linker, L, may be the same or different.

In some embodiments, the self-immolative linker, L, may comprise a spacer group between the terminating moiety, T, and the self-immolative group(s) as defined herein (i.e., the aminobenzyl carbamate, aminobenzyl ether, dithiobenzyl carbamate and/or dithiobenzyl ether group(s)). In such embodiments, the compound of the invention may suitably be of the formula Xₘ—(Y-L′ₙ-S″-T), wherein each of X, m, Y and T and are as defined herein; L′ is a self-immolative group (such as a methylene carbamate, aminobenzyl carbamate, aminobenzyl carbonate, aminobenzyl ether, dithiobenzyl carbamate, dithiobenzyl carbonate or dithiobenzyl ether group as defined herein); n is 1 to 5, for example 1 to 4, for example 1 to 3, for example 1 or 2, for example 1; and S″ is a spacer group.

As described above, when m is 1, the cleavable group, Y, may comprise one or more stretcher unit(s), S, between X and the cleavable group, Y. In such embodiments, the compound of the invention may be of the formula X—(Sₓ—Y-L′ₙ-S″-T), wherein each of X, S, x, Y, L′, n and T are as defined herein and S″ is a spacer group.

Advantageously, the inclusion of an appropriate spacer group, S″, may increase stability and/or hydrophilicity and/or decrease steric hindrance with the cleavable group, Y, and/or self-immolative linker, L.

The spacer group, S″, may be self-immolative.

The spacer group, S″, may be represented by the formula:

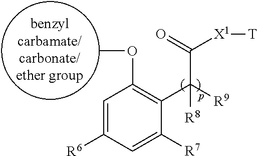

wherein T is a terminating moiety as defined herein; X₁ is O, NH, NR or S, wherein R is a C₁-C₆ alkyl group; p is 1 or 2; and each of R⁶, R⁷, R⁸ and R⁹ are independently H or C₁-C₈alkyl. Such spacer groups are described in, for example, U.S. Pat. No. 6,210,345, which is herein fully incorporated by reference.

The spacer group may comprise a self-cyclizing group. Self-cyclizing groups are suitably able to undergo a self-cyclization reaction upon cleavage of the cleavable group, Y.

The self-cyclizing spacer group may comprise a group of the formula —(CH₂)ₙ—NR—C(O)—O—, wherein n is 2 to 10, for example 2 to 6, for example 2 to 4, for example 2 or 3, for example 2; and each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group.

When the self-immolative linker, L, comprises an aminobenzyl carbamate, aminobenzyl carbonate, dithiobenzyl carbamate or dithiobenzyl carbonate group and a self-cyclizing spacer group of the formula —(CH₂)ₙ—NR—C(O)—O—, wherein each of n and R are as defined herein, the self-immolative linker, L, may be of the formula:

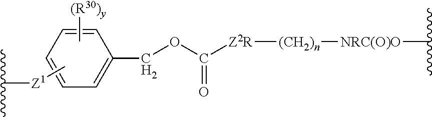

wherein each of R³⁰, y, R and n are as defined herein; Z¹ is —NH— or —S—S—; and Z² is N or O.

When the self-immolative linker, L, comprises an aminobenzyl carbamate or dithiobenzyl carbamate group and a self-cyclizing spacer group of the formula —(CH₂)ₙ—NR—C(O)—O—, the self-immolative linker, L, suitably comprises an alkylenediamine carbamate group of the formula —N*R—(CH₂)ₙ—NR—C(O)—O—, wherein n is 2 to 10, for example 2 to 6, for example 2 to 4, for example 2 or 3, for example 2; each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group; and the N atom marked with an asterisk is from the carbamate group of the aminobenzyl carbamate or dithiobenzyl carbamate group.

When the self-immolative linker, L, comprises an alkylenediamine carbamate group, the compound of the invention may be of formula Xₘ—(Y-L-NR—(CH₂)ₙ—NR—C(O)—O-T), wherein each of X, m, S, Y, L and T are as defined herein; x is 0, 1 or 2; n is 2 to 10, for example 2 to 6, for example 2 to 4, for example 2 or 3, for example 2; and each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group.

As described above, when m is 1, the cleavable group, Y, may comprise one or more stretcher unit(s), S, between X and the cleavable group, Y. In such embodiments, the compound of the invention may be of the formula X—(Sₓ—Y-L′ₙ-NR—(CH₂)ₙ—NR—C(O)—O-T), wherein each of X, S, x, Y, L′, n and T are as defined herein; n is 2 to 10, for example 2 to 6, for example 2 to 4, for example 2 or 3, for example 2; and each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group.

n in relation to the alkylenediamine carbamate group may be 2. As, such, the self-immolative linker, L, may comprise an ethylenediamine carbamate group (i.e., —NR—CH₂—CH₂—NR—C(O)—O—, wherein each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group).

The self-immolative linker, L, may comprise N,N′-dimethyl ethylenediamine carbamate (i.e., —N(CH₃)—CH₂—CH₂—N(CH₃)—C(O)—O—).

The self-cyclizing group may undergo a self-cyclization reaction upon cleavage of the cleavable group, Y, and self-immolation of the self-immolative linker, L, resulting in the release of a heterocyclic compound. The released heterocyclic compound may be a volatile organic compound. For example, when the self-immolative linker, L, comprises an ethylenediamine carbamate group, the self-cyclization reaction product, released upon cleavage of the cleavable group, Y, and self-immolation of the self-immolative linker, L, may suitably be of the formula:

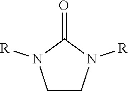

wherein each R is hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group.

Thus, the self-cyclization reaction product may comprise 2-imidazolidinone and/or a 1,3-di(C₁-C₆ alkyl)-2-imidazolidonone, for example 1,3-di(C₁-C₄ alkyl)-2-imidazolidonone, for example 1,3-di(C₁-C₃ alkyl)-2-imidazolidonone, for example 1,3-di(C₁-C₂ alkyl)-2-imidazolidonone, or for example 1,3-dimethyl-2-imidazolidonone. Such compounds are known to be volatile organic compounds.

When the self-immolative group comprises an aminobenzyl carbamate, aminobenzyl carbonate, dithiobenzyl carbamate and/or dithiobenzyl carbonate group, the self-immolative linker, L, may covalently link the terminating moiety, T, (via the carbamate or carbonate group) to the cleavable group, Y, (via a peptide, disulphide and/or glucuronide bond, for example) to provide a tripartite molecule. Such a tripartite molecule may suitably be stable in the absence of a target enzyme and/or other suitable cleavage conditions. However, upon action of a target enzyme and/or any other suitable cleavage conditions, the peptide, disulphide and/or glycosidic bond will suitably be cleaved. Upon such cleavage, whether by enzymatic or other means, e.g., hydrolysis, for example, the self-immolative linker, L, suitably undergoes a spontaneous reaction that causes cleavage of the carbamate or carbonate group. Under such conditions, carbon dioxide (CO₂) is suitably released. The reaction may proceed by 1,6- or 1,4-elimination.

When the self-immolative linker, L, comprises an aminobenzyl carbamate, carbonate or ether group, cleavage of the self-immolative group, L, may also release iminoquinone methide. The iminoquinone methide may form aminobenzyl alcohol upon reaction with water. In some embodiments, the iminoquinone methide may be para-iminoquinone methide and/or the aminobenzyl alcohol may be 4-aminobenzyl alcohol. In some embodiments, the iminoquinone methide may be ortho-iminoquinone methide and/or the aminobenzyl alcohol may be 1-aminobenzyl alcohol. Suitably, iminoquinone methide and/or aminobenzyl alcohol may be released upon a 1,6-elimination reaction.

When the self-immolative group comprises an aminobenzyl ether or dithiobenzyl ether group, the self-immolative linker, L, may covalently link the terminating moiety, T, (via the ether group) to the cleavable group, Y, (via a peptide, disulphide and/or glucuronide bond, for example) to provide a tripartite molecule. Such a tripartite molecule may suitably be stable in the absence of a target enzyme and/or other suitable cleavage conditions. However, upon action of a target enzyme and/or any other suitable cleavage conditions, the peptide, disulphide and/or glycosidic bond will suitably be cleaved. Upon such cleavage, whether by enzymatic or other means, e.g., hydrolysis, for example, the self-immolative linker, L, suitably undergoes a spontaneous reaction that causes cleavage of the ether group.

The self-immolative linker, L, may comprise a reporter molecule or a precursor thereof. The reporter molecule or precursor thereof may be released from the self-immolative linker, L, upon cleavage of the cleavable group, Y, and spontaneous degradation of the self-immolative linker, L.

For example, when the self-immolative linker, L, comprises an aminobenzyl carbamate, aminobenzyl carbonate, dithiobenzyl carbamate and/or dithiobenzyl carbonate group, cleavage of the cleavable group, Y, suitably triggers spontaneous degradation of the self-immolative linker, L, and the release of carbon dioxide (CO₂). In such embodiments, the reporter molecule may comprise carbon dioxide (CO₂).

For example, when the self-immolative linker, L, comprises an aminobenzyl carbamate, aminobenzyl carbonate or aminobenzyl ether group, cleavage of the cleavable group, Y, suitably triggers spontaneous degradation of the self-immolative linker, L, and the release of iminoquinone methide and/or aminobenzyl alcohol. In such embodiments, the reporter molecule may comprise carbon dioxide (CO₂), iminoquinone methide and/or aminobenzyl alcohol. Preferably, the reporter molecule may comprise carbon dioxide (CO₂).

For example, when the self-immolative linker, L, comprises an aminobenzyl carbamate, aminobenzyl carbonate, dithiobenzyl carbamate and/or dithiobenzyl carbonate group and a self-cyclizing spacer group, cleavage of the cleavable group, Y, suitably triggers spontaneous degradation of the self-immolative linker, L, and self-cyclization of the spacer group resulting in the release of carbon dioxide (CO₂) and a self-cyclization reaction product, for example a heterocyclic compound. In such embodiments, the reporter molecule may suitably comprise carbon dioxide (CO₂) and/or the self-cyclization reaction product, for example a heterocyclic compound. In such embodiments, the reporter molecule may additionally or alternatively comprise iminoquinone methide and/or aminobenzyl alcohol. Preferably, the reporter molecule may comprise carbon dioxide (CO₂).

In some embodiments, the self-immolative linker, L, may be isotopically labelled. It will be appreciated by a person skilled in the art that in such embodiments the self-immolative linker, L, should be isotopically labelled at one or more suitable atom(s), as appropriate, such that the released reporter molecule is isotopically labelled. For example, when the self-immolative linker, L, is an aminobenzyl carbamate, aminobenzyl carbonate, dithiobenzyl carbamate and/or dithiobenzyl carbonate group, the self-immolative linker, L, may be isotopically labelled at one or more suitable atom(s), as appropriate, such that the released carbon dioxide is isotopically labelled. When the self-immolative linker, L, comprises an aminobenzyl carbamate, aminobenzyl carbonate or aminobenzyl ether group, cleavage of the cleavable group, Y, the self-immolative linker, L, may be isotopically labelled at one or more suitable atom(s), as appropriate, such that the released carbon dioxide, iminoquinone methide and/or aminobenzyl alcohol is isotopically labelled. One or more of the carbon dioxide, iminoquinone methide and/or aminobenzyl alcohol may be isotopically labelled.

In some embodiments, the self-immolative linker, L, may be of the formula:

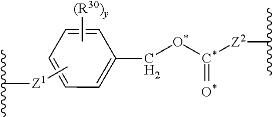

wherein each of R³⁰ and y are as defined herein; Z¹ is —NH— or —S—S—; Z² is N or O; and one or more of the atoms marked with an asterisk are isotopically labelled. In such embodiments, the reporter molecule is suitably isotopically labelled carbon dioxide.

In some embodiments, the self-immolative linker. L, may be of the formula:

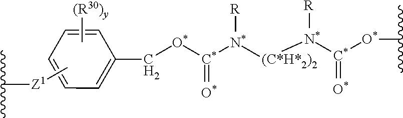

wherein each of R³⁰ and y are as defined herein; Z¹ is —NH— or —S—S—; n is 2 to 10, for example 2 to 6, for example 2 to 4, for example 2 or 3, for example 2; and each R is independently hydrogen or a C₁-C₆ alkyl group, for example hydrogen or a C₁-C₄ alkyl group, for example hydrogen or a C₁-C₃ alkyl group, for example hydrogen or a C₁-C₂ alkyl group, for example hydrogen or a methyl group, or for example a methyl group; and one or more of the atoms marked with an asterisk and/or any of the atoms of the R groups are isotopically labelled. In such embodiments, the reporter molecule is suitably isotopically labelled carbon dioxide, 2-imidazolidinone and/or a 1,3-di(C₁-C₆ alkyl)-2-imidazolidonone.

It will be appreciated by the skilled person that when iminoquinone methide and/or aminobenzyl alcohol are released upon cleavage of the cleavable group, Y, and spontaneous degradation of the self-immolative linker, L, one or more of the atoms of the self-immolative linker, L, may additionally or alternatively be isotopically labelled, as appropriate, such that the iminoquinone methide and/or aminobenzyl alcohol are isotopically labelled.

As described herein, one or more compounds may be released from the self-immolative linker, L, upon cleavage of the cleavable group, Y. In some embodiments, at least one of the released compounds may be a reporter molecule (i.e., may be an isotopically labelled VOC). In some embodiments, none of the released compounds may be a reporter molecule. For example, the released compounds may be unlabelled and/or may not be a VOC. In such embodiments, the released compounds may nevertheless be used as an additional means of detection (on the proviso that a reporter molecule falling within the scope of the invention is also released from the compound, from the terminating moiety, T, for example). For example, one or more of the released compounds may be detected along with one or more of the reporter molecules described herein.

As described herein, the compound may comprise a further cleavable group, Y′. In such embodiments where the compound comprises a further cleavable group, Y′, and the self-immolative linker, L, comprises an aminobenzyl carbamate group, an aminobenzyl carbonate group, an aminobenzyl ether group, a dithiobenzyl carbamate group, a dithiobenzyl carbonate group and/or a dithiobenzyl ether group, the compound may be of the formula:

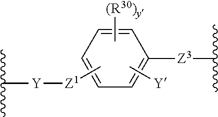

wherein each of Y, Y′, Z′ and R³⁰ are as defined herein; y′ is an integer from 0 to 3; and Z³ is a methylene carbamate (—CH₂—O—C(O)—NH—), methylene carbonate (—CH₂—O—C(O)—O—) or methylene ether (—CH₂—O—) group. In some embodiments, Y may be a peptide. In some embodiments, Y′ may be a glucuronide group. In some embodiments, Y may be a peptide and Y′ may be a glucuronide group.

In some embodiments, the further cleavable group, Y′ may be unlabelled. In some embodiments, the further cleavable group, Y′, may be isotopically labelled. For example, the further cleavable group, Y′, may be a glucuronide group that is isotopically labelled at one or more atom(s) thereof. Advantageously, the use of isotopically labelled further cleavable groups, Y′, may provide a further means of detection.

### Terminating Moiety, T

T is a terminating moiety. T may be any suitable terminating moiety.

Preferably, the terminating moiety, T, may comprise a leaving group. A person skilled in the art will know that a leaving group is an atom or group of atoms that is able to detach from the main or residual part of a molecule during a reaction or elementary step of a reaction. Typically, a leaving group is a group that departs with a pair of electrons in heterolytic bond cleavage.

The terminating moiety may comprise hydrogen, a C1-C10 alkyl group, a C2-C10 alkenyl group, a C2-C10 alkynyl group, an aryl group, a therapeutically active agent and/or a molecular label.

Preferably, the terminating moiety T, may comprise a C1-C10 alkyl group and/or a C2-C10 alkenyl group.

The terminating moiety, T, may comprise a C1-C10 alkyl group, for example a C1-C6 alkyl group, for example a C1-C4 alkyl group, for example a C1-C2 alkyl group, for example an ethyl group.

The terminating moiety, T, may comprise a C2-C10 alkenyl group, for example a C2-C8 alkenyl group, for example a C2-C6 alkenyl group, for example a C4-C6 alkenyl group, for example a hexenyl group, or for example, trans-2-hexenol.

In some embodiments, the terminating moiety, T, may comprise a therapeutically active agent. Examples of suitable therapeutically active agents include, but are not limited to, drugs, enzymes and/or toxins. The therapeutically active agent may comprise a toxin, for example a cytotoxic radionuclide, chemical toxin or protein toxin.

In some embodiments, the terminating moiety may comprise a molecular label. By “molecular label”, and like terms as used herein, is meant a group that is operable to aid the detection of the compound. Detection of the compound may be ex vivo and/or in vivo. Examples of suitable molecular labels include, but are not limited to, fluorescent molecules, β-galactosidase, luciferase molecules, chemical dyes, fluorophores and/or radioisotopes.

The terminating moiety, T, may comprise a reporter molecule or a precursor thereof. The reporter molecule or precursor thereof may be released from the terminating moiety, T, upon cleavage of the cleavable group, Y. Cleavage of the cleavable group, Y, suitably triggers spontaneous degradation of the self-immolative linker, L, resulting in release of the terminating moiety, T. For example, when the self-immolative linker, L, comprises an aminobenzyl carbonate and/or dithiobenzyl carbonate group and the terminating moiety, T, is a C1-C10 alkyl group, cleavage of the cleavable group, Y, suitably triggers spontaneous degradation of the self-immolative linker, L, resulting in the release carbon dioxide (CO₂) and a C1-C10 alkanol. In such embodiments, the reporter molecule suitably comprises carbon dioxide (CO₂) and/or a C1-C10 alkanol.

For example, when the self-immolative linker, L, comprises an aminobenzyl carbamate and/or dithiobenzyl carbamate group and the terminating moiety, T, is a C1-C10 alkyl group, cleavage of the cleavable group, Y, suitably triggers spontaneous degradation of the self-immolative linker, L, resulting in the release carbon dioxide (CO₂) and a C1-C10 alkylamine. In such embodiments, the reporter molecule suitably comprises carbon dioxide (CO₂) and/or a C1-C10 alkylamine.

For example, when the self-immolative linker, L, comprises an aminobenzyl ether and/or a dithiobenzyl ether group and the terminating moiety, T, is a C1-C10 alkyl group, cleavage of the cleavable group, Y, suitably triggers spontaneous degradation of the self-immolative linker, L, and the release of ethanol. In such embodiments, the reporter molecule suitably comprises a C1-C10 alkanol.

In some embodiments, the terminating moiety, T, may be isotopically labelled. It will be appreciated by a person skilled in the art that in such embodiments the terminating moiety, T, should be isotopically labelled at one or more suitable atom(s), as appropriate, such that the released reporter molecule is isotopically labelled. For example, when terminating moiety, T, is an alkyl, alkenyl, alkynyl or aryl group, the terminating moiety, T, may be isotopically labelled at one or more suitable atom(s), as appropriate, such that the released alkanol, alkenol, alkynol or aryl alcohol group is isotopically labelled.

In some embodiments, the terminating moiety, T, may be unlabelled.

In some embodiment, the self-immolative linker, L, and terminating moiety, T, may be of the formula:

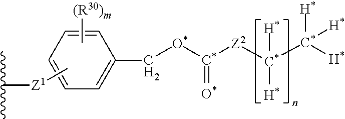

wherein each of Z¹, R³⁰, and m are as defined herein; Z² is O* or N*H*; n is 0-9; and one or more of the atoms marked with an asterisk are isotopically labelled. In such embodiments, the reporter molecule may suitably be isotopically labelled carbon dioxide, an isotopically labelled alkylamine and/or isotopically labelled alkanol. It will be appreciated by a person skilled in the art that in such embodiments one or each of the carbon dioxide, alkylamine and/or alkanol may be isotopically labelled.

### Reporter Molecule

At least one of the self-immolative linker, L, and terminating moiety, T, comprise a reporter molecule or a precursor thereof.

The term “precursor thereof” in relation to the reporter molecule, refers to moieties that may be further processed, such as further metabolised, upon release thereof. “Precursor thereof” should also be construed as including moieties wherein one or more additional atoms are provided by external sources, i.e., moieties others than the compound of the invention, upon release of the reporter molecule. For example, when the reporter molecule is ethanol, the hydrogen atom of the hydroxyl group may suitably be provided by a water molecule (such as upon hydrolysis, for example) Reference herein to “reporter molecule” should be interpreted to include precursors thereof, unless specified otherwise.

The reporter molecule is operable to be released upon cleavage of the cleavable group, Y (and spontaneous degradation of the self-immolative linker, L, as described herein).

The reporter molecule, upon release, is a volatile compound.

The reporter molecule is isotopically labelled.

The reporter molecule may be any suitable compound (on the proviso that, upon release, it is a volatile compound and is isotopically labelled).

The reporter molecule may be a volatile organic compound (VOC). The reporter molecule when attached to the compound of the invention is not volatile, however once the reporter molecule is released from the compound under the conditions described herein it is suitably volatile.

The term volatile compound refers to any compound of carbon which participates in atmospheric photochemical reactions. The term VOC refers to any compound of carbon, excluding carbon monoxide, carbon dioxide, carbonic acid, metallic carbides or carbonates and ammonium carbonate, which participates in atmospheric photochemical reactions but excluding carbon monoxide, carbon dioxide, carbonic acid, metallic carbides or carbonates, and ammonium carbonate. Generally, volatile compounds and/or VOCs are defined as organic chemical compounds whose composition makes it possible for them to evaporate under normal indoor atmospheric conditions of temperature and pressure. Since the volatility of a compound is generally higher the lower its boiling point temperature, the volatility is sometimes defined and classified by their boiling points. Volatile compounds and/or VOCs are compounds that are secreted by the human body into gas fluids, including for example breath, skin emanations and others. In one embodiment, a volatile compound and/or VOC is any organic compound having an initial boiling point less than or equal to about 250° C. measured at a standard atmospheric pressure of about 101.3 kPa.

The reporter molecule is preferably a volatile compound that may be secreted in exhaled breath. The reporter molecule may suitably be present in a sample of exhaled breath in sufficient amounts to enable it to be detected (for example, in amounts above “normal” or “background” levels).

The reporter molecule may be an exogenous volatile compound.

The term “exogenous volatile organic compound” refers to compounds that are not produced naturally by a subject. The reporter molecule may not be present naturally in the exhaled breath of a subject. This ensures that any readings are not contaminated by endogenous molecules, such as endogenous volatile compounds, that are naturally produced and can be found in exhaled breath. It will be appreciated by a person skilled in the art that isotopically labelled molecules are not typically endogenous, i.e., are not typically naturally secreted in exhaled breath.

The reporter molecule may comprise carbon dioxide, a C1-C10 alkanol, a C2-C10 alkenol, a C1-C10 alkylamine, 2-imidazolidinone, a 1,3-di(C1-C6 alkyl)-2-imidazolidonone, iminoquinone methide and/or aminobenzyl alcohol.

The reporter molecule may comprise carbon dioxide.

The reporter molecule may comprise a C1-C10 alkanol, for example a C1-C6 alkanol, for example a C1-C4 alkanol, for example a C1-C2 alkanol, or for example ethanol.

The reporter molecule may comprise a C2-C10 alkenol, for example a C2-C8 alkenol, for example a C2-C6 alkanol, for example a C4-C6 alkanol, for example hexenol, or for example trans-2-hexenol.

The reporter molecule may comprise a C1-C10 alkylamine, for example a C1-C6 alkylamine, for example a C1-C4 alkylamine, for example a C1-C2 alkylamine, or for example ethylamine.

The reporter molecule may comprise 2-imidazolidinone and/or a 1,3-di(C1-C6 alkyl)-2-imidazolidonone, for example 1,3-di(C1-C4 alkyl)-2-imidazolidonone, for example 1,3-di(C1-C3 alkyl)-2-imidazolidonone, for example 1,3-di(C1-C2 alkyl)-2-imidazolidonone, or for example 1,3-dimethyl-2-imidazolidonone.

The reporter molecule may comprise iminoquinone methide and/or aminobenzyl alcohol.

The reporter molecule may comprise carbon dioxide and/or a C1-C10 alkanol, for example ethanol.

The reporter molecule is isotopically labelled. The isotopic label may be any suitable label. For example, the label may be 12C, 13C, 14C, 2H (or D), 14N, 17O and/or 18O. For the avoidance of doubt, 2H is deuterium and may be referred to as “2H”, “D” or “deuterium” interchangeably herein.

The isotopic label may be 13C, 14C, 2H, 17O and/or 18O.

When the reporter molecule is isotopically labelled carbon dioxide, the isotopic label may be 13C/14C and/or 17O/18O, for example 13C and/or 17O/18O.

When the reporter molecule is an isotopically labelled C1-C10 alkanol and/or C1-C10 alkenol, the isotopic label may be 13C/14C, 2H and/or 17O/18O, for example 2H.

It will be appreciated by a person skilled in the art that a small proportion of natural carbon is 13C (approximately 1%). As such, when the reporter molecule is carbon dioxide that is isotopically labelled with only 13C, there may naturally be a background level of the same molecule present in the exhaled breath of the subject. In such embodiments, a sample of exhaled breath may be taken from the subject before and after administration of the compound of the invention.

The reporter molecule may comprise 13C and/or 14C labelled carbon dioxide.

The reporter molecule may comprise 14C labelled carbon dioxide. Advantageously, the use of 14C labelled carbon dioxide as the reporter molecule allows for sensitive detection (on the basis that the natural abundance of 14C is only 1 part per trillion, i.e., 1.0×10⁻¹⁰%, compared to approximately 1.1% for 13C).

The reporter molecule may comprise carbon dioxide labelled with 13C/14C and 17O/18O, for example with 13C and 17O/18O. For example, the carbon dioxide may be labelled with 13C at the carbon atom and 17O/18O at one or both of the oxygen atoms. Advantageously, the use of more than one label means that the carbon dioxide may be more easily detected above background levels (because such combinations of carbon/oxygen isotopes are rarer in nature).

The reporter molecule may comprise a 13C/14C, 2H and/or 17O/18O labelled alkanol, for example ethanol.

The reporter molecule may comprise D5-ethanol.

The reporter molecule may comprise at least two different isotopic labels. For example, the reporter molecule may comprise a 13C/14C, 2H and/or 17O/18O labelled alkanol, for example ethanol, wherein at least two different atoms are labelled (for example, C and H, C and O, etc.). Advantageously, the use of more than one label means that the reporter molecule may be more easily detected.

The reporter molecule may comprise carbon dioxide and a C1-C10 alkanol, for example ethanol. Advantageously, the use of more than one reporter molecule means that the reporter molecules may be more easily detected.

As described above, in some embodiments, n may be at least 2. In such embodiments, each of the Y, L and T groups may be the same or different. For example, each cleavable group, Y, may be the same or may be different. Preferably, each cleavable group, Y, may be different, i.e., each cleavable group, Y, may be operable to be cleaved by a different protease. In such embodiments, the same or different reporter molecule may be released from each —(Y-L-T) group. Preferably, a different reporter molecule may be released from each —(Y-L-T) group. In such embodiments, it is an advantage that the activity of two or more proteases, for example, can be monitored separately (i.e., via the detection of the different reporter molecules).

### Composition

The invention also relates to a composition comprising a compound of the invention. The composition may comprise a compound of the invention and optionally a pharmaceutically acceptable carrier or excipient.

The composition may be an inhalable composition.

The composition may comprise one or more compounds of the invention (as described herein). The composition may comprise only one compound of the invention (as described herein). The composition may comprise two or more compounds of the invention (as described herein).

When the composition comprises two or more compounds of the invention, each compound may suitably comprise a cleavable group, Y, that is operable to be cleaved under different conditions. For example, each compound may comprise a cleavable group, Y, that is operable to be cleaved by a different protease. In such embodiments, the activity of each protease can be detected and/or monitored separately. As such, suitably, each compound may also comprise a different reporter molecule. In such embodiments, the activity of each protease can be detected and/or monitored separately via the detection of a different reporter molecule in the exhaled breath of a subject, for example.

The pharmaceutically acceptable carrier or vehicle can be particulate, so that the compositions are, for example, in tablet or powder form. The term “carrier” refers to a diluent, adjuvant or excipient, with which compound of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and colouring agents can be used. When administered to a human or animal subject, such as a human subject, the compounds, compositions and/or pharmaceutically acceptable carriers may suitably be sterile. Water is a preferred carrier when the compound of the present invention is to be administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The composition can be in the form of a liquid, e.g., a solution, emulsion or suspension. The liquid can be useful for delivery by injection, infusion (e.g., IV infusion) or sub-cutaneously.

When intended for oral administration, the composition may be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

As used herein, the term “effective amount” means an amount of compound that, when administered to a cell, tissue, or subject, is effective to achieve the desired diagnostic and/or therapeutic effect under the conditions of administration. The term “effective amount” of a composition, as used herein, is intended to denote a non-lethal but sufficient amount of the composition to provide the desired effect. For example, in order to detect the reporter molecule, and thus diagnose a disorder or infection, an effective amount is the one which enables a sufficient amount of the reporter molecule to be detected such that a diagnosis can be made. When the compound of the invention comprises a therapeutically active agent, i.e., when the terminating moiety, T, comprises a therapeutically active agent, in order to elicit a favourable response in a subject when treating a disorder or infection, the effective amount is the one which eliminates or diminishes the symptoms associated with the disorder, e.g., by eliminating or reducing the size of a tumour, for example. An effective amount may be determined by one of ordinary skill in the art, using routine experimentation.

In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease may be taken into account.

The amount may be at least about 0.01% of the compound by weight of the composition. When intended for oral administration, this amount can be varied to range from about 0.1% to about 80% by weight of the composition, such as from about 4% to about 50% by weight of the composition. Compositions of the present invention can be prepared so that a parenteral dosage unit contains from about 0.01% to about 2% by weight of the compound.

For administration by injection, the composition may comprise from about 0.1 mg/kg to about 250 mg/kg of the subject's body weight, for example, between about 0.1 mg/kg and about 20 mg/kg of the subject's body weight, for example about 1 mg/kg to about 10 mg/kg of the subject's body weight. The composition may be administered at a dose of about 1 to 30 mg/kg, e.g., about 5 to 25 mg/kg, about 10 to 20 mg/kg, about 1 to 5 mg/kg, or about 3 mg/kg.

The compound and/or composition may be administered once or multiple times. For example, the compound and/or composition may be administered at an initial dose, followed by one or more secondary doses.

### Kit

The reporter molecule is suitably detected in a sample from a subject. The sample may be any suitable sample. Samples may include, but are not limited to, nasal and/or nasopharyngeal swabs, washes or aspirates, nasal mucosal fluid, oral and/or oropharyngeal swabs, washes or aspirates, breath such as exhaled breath, biological fluids such as serum, saliva plasma, whole blood, peripheral blood, capillary blood, sputum and cough secretions, isolated and/or separated cells or cell populations, cultured cells of any origin and cell extracts, as well as tissue material such as fresh or frozen biopsies and formalin-fixed or paraffin-embedded biopsies.

The sample may be a biological fluid, for example blood (including whole, peripheral and/or capillary blood) or breath, such as breath, or even exhaled breath.

Thus, the invention also relates to a kit comprising the compound and/or the composition of the invention and a device for capturing a breath sample from a subject.

The breath sample may be exhaled breath.

The device for capturing breath may be a sampling bag with or without a collection tube or analysis fitting attached.

The device for capturing breath may be as described in WO2017/187120 or WO2017/187141 (which are fully incorporated herein). The device in WO2017/187120 comprises a mask portion which, in use, is positioned over a subject's mouth and nose, so as to capture breath exhaled from the subject. The exhaled breath samples are fed into tubes containing a sorbent material, to which the compounds of interest adsorb. After sufficient sample has been obtained, the sorbent tubes are removed from the sampling device and the adsorbed compounds desorbed (typically by heating) and subjected to analysis to identify the presence and/or amount of any particular compounds or other substances of interest. The preferred analytic technique is field asymmetric ion mobility spectroscopy (abbreviated as “FAIMS”). The method in WO2017/187141 is a refinement of the method described in WO2017/187120. In WO2017/187141, it is taught to use breath sampling apparatus substantially of the sort described in WO2017/187120, but in a way such as to selectively sample desired portions of a subject's exhaled breath, the rationale being that certain biomarkers or other analytes of interest are relatively enriched in one or more fractions of the exhaled breath, which fractions themselves are relatively enriched in air exhaled from different parts of the subject's body (e.g. nostrils, pharynx, trachea, bronchioles, alveoli etc).

It can be desirable to administer the composition by injection, intravenous injection, inhalation, or infusion. As such, the kit may optionally comprise a device which includes an injector pen. For example, the compound and/or composition may be provided as a pre-filled syringe or other self-administration device.

The kit may optionally contain other components, packaging, instructions and/or material to aid the detection of the reporter molecule.

The kit may include a compound and/or composition of the invention packaged in lyophilized form or packaged in an aqueous medium.

The compound and/or composition of the invention may be administered as the sole active ingredient or in combination with one or more other agent, such as therapeutic agent. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes and radioisotopes. An antibody molecule includes a full antibody or fragment thereof as mentioned elsewhere herein, e.g., a Fab, F(ab′)2, Fv, a single chain Fv fragment (scFv) or a single domain antibody, for example a VH domain, or antibody mimetic protein.

One or more compound and/or composition as described herein may be used in combination with an existing therapy or therapeutic agent, for example an anti-viral compound, a drug, such as an anti-cancer drug, an antibody therapy, a vaccine, an immunomodulator or an anti-inflammatory. An existing therapy or therapeutic agent may be administered concurrently or sequentially with one or more compounds and/or compositions of the invention.

### Method

The invention also relates to a method for the detection or prognosis of a disease.

The term “disease” as used herein is used interchangeably with “disorder” and includes infections.

The term “detection” or “detecting” is used herein in the broadest sense to include both qualitative and quantitative measurements of the reporter molecule. Detecting includes identifying the mere presence of the reporter molecule in a sample as well as determining whether the reporter molecule is present in the sample at detectable levels. Detecting may be direct or indirect.

When the compound comprises a therapeutically active agent, i.e., when the terminating moiety comprises a therapeutically active agent, the invention may further relate to a method for the treatment of a disease. As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the subjects, e.g., human patients, being treated. Many medical treatments are effective for some, but not all, patients that undergo the treatment.

The disease may be any suitable disease.

The disease may be a liver disease. The liver disease may be selected from non-alcoholic steatohepatitis (NASH), non-alcoholic fatty liver disease (NAFLD) or nonalcoholic steatohepatitis (HCC).

The disease may be a cancer.

The cancer may be selected from lung cancer, breast cancer, ovarian cancer, bowel cancer, prostate cancer, bladder cancer, colorectal cancer, pancreas carcinoma, kidney cancer, renal cancer, leukaemias, multiple myeloma, lymphomas (e.g., Hodgkin's disease and non-Hodgkin's Lymphoma), brain cancer and other CNS and intracranial tumours cancer, head and neck cancer, oesophageal cancers, solid tumours such as sarcoma and carcinomas, mesothelioma, osteosarcoma, endometrial cancer or melanoma.

The cancer may be a lung cancer. Exemplary types of lung cancer include, but are not limited to, small cell lung cancer (SCLC), non-small cell lung cancer (NSCLC), which includes squamous cell carcinoma, and adenocarcinoma. Other subtypes of lung cancer include bronchioloalveolar carcinoma, large cell carcinoma, carcinoid, adenoid cystic carcinoma, cylindroma, and mucoepidermoid carcinoma. In one embodiment, lung cancers are staged according to stages I-IV, with I being an early stage and IV being the most advanced. The methods of the invention would be of particular benefit to patients with surgically resected stage I or II non-squamous NSCLC. The current standard of care for most stage I non-squamous NSCLC is lobectomy and mediastinal lymph node dissection, without adjuvant chemotherapy. Better identification of good prognosis patient subsets might allow lesser surgical procedures to be employed with equal survival potential. Conversely, stage I subsets with a poor prognosis could be selected for treatment with adjuvant chemotherapy to reduce the risk of distant recurrence using current standard-of-care agents. Furthermore, patients identified to have a poor prognosis might also be considered for inclusion into clinical trials testing novel approaches and new therapeutic agents. Considering the current limitations of chemotherapy in stage I disease, a bioassay that is both prognostic and predictive of chemotherapy benefit would be especially beneficial. Lastly, stage I non-squamous NSCLC is likely to be of increasing importance in the future.

Patients with stage II NSCLC are currently recommended to undergo adjuvant chemotherapy after attempt at curative resection. The documented benefit of chemotherapy for these patients in terms of absolute improvement in 5-year survival, however, is small. As a result, many patients forego chemotherapy, particularly as they recover from their attempt at curative surgery. A diagnostic method that can better assign risk of recurrence to stage II patients may therefore improve compliance with current standard-of-care recommendations for adjuvant therapy in patients found to be at higher risk of recurrence. In a controlled, experimental setting, therapy may even be withheld from patients found to be at the lowest risk for recurrence even in stage II.

The method comprises administering a compound and/or composition of the invention to a subject and measuring the concentration of the released reporter molecule in the exhaled breath of the subject.

The term “subject” or “patient” refers to an animal which is the object of diagnosis, treatment, observation, or experiment. By way of example only, a subject includes, but is not limited to, a mammal, including, but not limited to, a human or a non-human mammal, such as a non-human primate, murine, bovine, equine, canine, ovine, or feline. The subject is preferably a human. The subject may be male or female. The subject may be an infant, a toddler, a child, a young adult, an adult or a geriatric. The subject may be a smoker, a former smoker or a non-smoker. The subject may have a personal or family history of cancer. The subject may have a cancer-free personal or family history. The subject may exhibit one or more symptoms of a disease, such as a cancer.

A sample of exhaled breath may be obtained from the subject for the purpose of diagnosing or screening the presence/absence of a disease state, such a cancer disease state, or making a prognosis as to the likelihood that the subject will develop a disease, such as a cancer.

The method may comprise the step of establishing a test subject value based on a concentration of the released reporter molecule.

The test subject value may be compared to one or more reference value and wherein a difference in the test subject value and a reference value indicates a likelihood of disease.

The reference value may be the value of a subject that has been diagnosed with a disease of interest.

The reference value may be the value of a healthy subject and/or may be the value of the subject prior to administration of the compound and/or composition of the invention.

The method may comprise the step of collection of a breath sample from said subject.

As used herein, a “healthy subject” is defined as a subject that does not have a diagnosable disease state, for example a diagnosable cancer disease state.

A “test subject value” is the value obtained in a test subject, i.e. a subject that is being assessed for a disease, for example cancer. The test value is the concentration of the reporter molecule that is measured in exhaled breath.

As used herein, “reference value”, “baseline” or “threshold value” means a value determined by performing the testing method on one or more, preferably a plurality of reference subjects. A reference subject can be a healthy subject or a subject diagnosed with a disease, for example cancer.

“Risk assessment” refers to the relative risk an individual faces with respect to mortality. For example, a prognosis providing a high-risk assessment for e.g. a 5-year mortality has a greater likelihood of mortality within 5 years than an individual having a low risk assessment for 5-year mortality. In one embodiment, the prognosis for long term mortality is “high risk,” e.g., high risk of mortality, “intermediate risk,” e.g., intermediate risk of mortality, or “low risk,” e.g., low risk of mortality. When the disease of interest is a cancer, the stage of cancer and the prognosis may be used to tailor a patient's therapy to provide a better outcome, e.g., systemic therapy and surgery, surgery alone, or systemic therapy alone. Risk assessment can be divided as desired, e.g., at the median, in tertiary groups, quaternary groups, and so on.

A “likelihood of a cancer disease state” means that the probability that the cancer disease state exists in the subject specimen is about 50% or more, for example 60%, 70%, 80% or 90%.

“Prognosis” refers, e.g., to overall survival, long term mortality, and disease-free survival. For example, long term mortality may refer to death within 5 years after diagnosis of a cancer, such as lung cancer.

“Cancer therapies” refers to chemotherapy, hormonal therapy, radiotherapy, immunotherapy, and biologic (targeted) therapy.

The method comprises the step of administering the compound to a subject. Administration may by any convenient route, including but not limited to oral, topical, parenteral, sublingual, rectal, vaginal, ocular, intranasal, pulmonary, intradermal, intravitrial, intramuscular, intraperitoneal, intravenous, subcutaneous, intracerebral, transdermal, transmucosal, or topical, particularly to the ears, nose, eyes, or skin or by inhalation. Parenteral administration includes, for example, intravenous, intramuscular, intraarterial, intraperitoneal, intranasal, rectal, intravesical, intradermal, topical or subcutaneous administration. Preferably, the compositions are administered orally, for example as a liquid, capsule or tablet, such as a slow release formulation. A skilled person would know that the route of administration depends on the disease of interest and the target antigen. For instance, if the target antigen is present in the gastrointestinal tract, oral administration is preferable, while in case of hepatic expression either oral or intravenous administration could constitute viable options. In the case of a lung disease, such as lung cancer, the enzyme can be administered via oral administration or inhalation.

The method comprises the step of detecting the presence or absence of the released reporter molecule in the exhaled breath of the subject.

The reporter molecule may suitably be released by cleavage of the cleavable linker, L. For example, the reporter molecule may be released due to cleavage of the cleavable linker, L, by an enzyme, such as a protease or glucuronidase, and/or by cleavage of a self-immolative group.

When the cleavable linker, L, is cleaved by an enzyme, the enzyme may be a disease specific enzyme.

A disease specific enzyme may be a cancer-specific enzyme. As used herein a “cancer-specific enzyme” is an enzyme that is selected from one or more of the following: the enzyme is absent in cancer tissue, but present in non-cancer tissue; the enzyme is present in cancer tissue, but absent in non-cancer tissue; the enzyme is differentially expressed in cancer tissue compared to non-cancer tissue or the enzyme is differentially active in cancer tissue compared to non-cancer tissue. For example, the enzyme may be expressed at a higher level in cancer tissue or lower level compared to expression in non-cancer tissue. Expression can be measured by techniques known in the art, for example by mRNA quantification or measuring cDNA. Non-cancer tissue refers for example to healthy tissue. The tissue may be from a specific organ, e.g., lung, colon, breast, prostate etc.

The enzyme may be localised to a different location in cancer tissue compared to non-cancer tissue, for example the enzyme may be present in the extracellular space of cancer tissue, whereas in non-cancer tissue the enzyme is not present in the extracellular space. A combination of tumour necrosis and release of lysosomal enzymes from macrophages and neutrophils can result in the presence of certain enzymes being present in the extracellular space of the tumour microenvironment. The exogenous substrate may be a substrate for an enzyme which is present in the extracellular space of solid tumours. The enzyme may be an enzyme which is an extracellular lysosomal enzyme. An “extracellular lysosomal enzyme” is an enzyme that has been released from the lysosome into the extracellular space.

The protease may be an endopeptidase and/or an exopeptidase. An endopeptidase is an enzyme which breaks peptide bonds other than terminal ones in a peptide chain. An exopeptidase is an enzyme that catalyzes the cleavage of the terminal or penultimate peptide bond; the process releases a single amino acid or dipeptide from the peptide chain.

The protease may comprise A20 (TNFa-induced protein 3); an abhydrolase domain-containing protein; an acrosin; an acylaminoacyl-peptidase; a disintegrin and metalloproteinase (ADAM), such as ADAM 12; a disintegrin and metalloproteinase with thrombospondin motifs (ADAMTS); an adipocyte-enh.-binding protein 1; an Afg3-like protein; an airway-trypsin-like protease; an aminoacylase; an aminopeptidase; an AMSH/STAMBP, an AMSH-LP/STAMBPL1; an angiotensin-converting enzyme; an anionic trypsin (II); an apolipoprotein (a); an archaemetzincin; an aspartoacylase; an aspartyl aminopeptidase; an ataxin-3; an ataxin-3 like; an ATP/GTP binding protein; an ATP23 peptidase; an autophagin; an azurocidin; or a combination thereof.

The protease may comprise a fibroblast activating protein (FAP).

The protease may comprise a beta lactamase, a beta-secretase; a bleomycin hydrolase; a brain serine proteinase 2; a BRCC36 (BRCA2-containing complex, sub 3); a calpain; or a combination thereof.

The protease may comprise a cysteine protease; a carboxypeptidase; a carnosine dipeptidase; a caspase; a casper/FLIP; a cathepsin, a cationic trypsin; a cezanne/OTU domain containing 7B; a cezanne-2; a CGI-58; a chymase; a chymopasin; a chymosin; a chymotrypsin; a coagulation factor; a collagenase; a complement serine protease; a complement component; a COPS6; a corin; a CSN5 (JAB1); a cylindromatosis protein; a cytosol alanyl aminopep.-like 1; a cytosol alanyl aminopeptidase; or a combination thereof.

The protease may comprise a DDI-related protease; a DECYSIN; a Deri-like domain; a DESCI protease; a desert hedgehog protein; a desumoylating isopeptidase; a dihydroorotase; a dihydropyrimidinase; a dihydropyrimidinase-related protein; a DINE peptidase; a dipeptidyl peptidase (DPP); a DJ-1; a DNA-damage inducible protein; a deubiquitinating enzyme (DUB); or a combination thereof.

The protease may comprise an enamelysin; an endopeptidase Clp; an endoplasmic reticulum metallopeptidase 1; an endothelin-converting enzyme; an enteropeptidase; an epidermis-specific SP-like; an epilysin; an epithelial cell transforming sequence 2 oncogene-like; an epitheliasin; an epoxide hydrolase; an epoxyde hydrolase related protein; an eukar. translation initiation F3SF; an eukar. translation initiation F3SH; or a combination thereof.

The protease may comprise a Factor VII activating protease, a FACE-1/ZMPSTE24, a FACE-2/RCE1, a family with sequence similarity 108, member A1, a family with sequence similarity 108; a furin; or a combination thereof.

The protease may comprise a gamma-glutamyl hydrolase, a gammaglutamyltransferase; a GCDFP15; a gelatinase; a Gln-fructose-6-P transamidase 1; a Gln-PRPP amidotransferase; a glutamate carboxypeptidase II; a glutaminyl cyclase; a glycosylasparaginase; a granzyme; a haptoglobin-1; or a combination thereof.

The protease may comprise a histone deacetylase (HDAC); a haptoglobin-related protein; a human airway trypsin (HAT)-like protease; a HAT-related protease, a heat shock 90 kDa protein; member 1/tumor rejection antigen (gp96); a hepatocyte growth factor; a hepsin; a HetF-like protease; a HGF activator; a hGPI8; a Hin-I/OTU domain containing 4; a homologue ICEY; a HP43.8KD, a HTRA serine protease; a hyaluronan-binding ser-protease; a implantation serine protease 2; an indian hedgehog protein; an insulysin; an intestinal serine protease 1; a josephin; or a combination thereof.

The protease may comprise a Kallikrein (KLK); a Kell blood-group protein; a KHNYN KH and NYN domain containing protease, a lactotransferrin; a legumain; a leishmanolysin-2; a leucyl aminopeptidase; a leucyl-cystinyl aminopeptidase; a leukotriene A4 hydrolase; a lysosomal carboxypeptidase A; a lysosomal Pro-X C-peptidase; or a combination thereof.

The protease may comprise a membrane metallo-endopeptidase (MME); a macrophage elastase; a macrophage-stimulating protein; a mammalian tolloid-like protein; a methione aminopeptidase; a marapsin; an aMBL associated serine protease; a mastin; a matrilysin; a matriptase; a membrane dipeptidase; a membrane-type mosaic Ser-protein; a meprin subunit; a mesoderm-specific transcript; a mesotrypsin; a methionyl aminopeptidase; a mitochondrial inner membrane protease 2; a mitochondrial intermediate peptidase; a mitochondrial proc.-protease; a mitochondrial signal peptidase; a matrix metalloproteinase (MMP); or a combination thereof.

The protease may comprise a NAALADASE; a napsin; a nardilysin; a nasal embryonic LHRH factor; a NEDD4 binding protein 1; a neprilysin; a neprilysin-2; a neurolysin; a neurotrypsin; a neutrophil elastase; a NLRP1 self-cleaving protein; a nuclear recept.-interacting protein 2; a nuclear recept.-interacting protein 3; a nucleoporin 98; a NYN domain and retroviral integrase containing protease; a NY-REN-60; an OMA1; an O-sialoglycoprotein endopeptidase; an osteoblast serine protease; an OTU domain containing protease; an otubain; an OTUD2/YOD1; an ovastacin; an oviductin-like/ovochymase-2; an ovochymase-like protease; or a combination thereof.

The protease may comprise a proteinase 3 (PRTN3); a papain; a PACE4 proprotein convertase; a pancreatic elastase; a pancreatic endopeptidase; a pappalysin; a paracaspase; a paraplegin; a pepsin; a PHEX endopeptidase; a PIDD auto-processing protein unit 1; a PIM endopeptidase; a pitrilysin metalloproteinase 1; a plasma Glu-carboxypeptidase; a plasma kallikrein; a plasma-kallikrein-like protease; a plasmin (plasminogen); a PM20D2 peptidase; a POH1/PSMD14; a polyserase; a Ppnx; a presenilin; a presenilin homolog; a presenilins-assoc. rhomboid like protease; a procollagen C-proteinase; a proliferation-association protein 1; a prolyl oligopeptidase; a prolyl oligopeptidase-like protease; a proprotein convertase; a prostasin; a proteasome alpha subunit; a proteasome beta subunit; a proteasome catalytic subunit; a protein C; a protein C-like protease; a protein Z; a proteinase 3; a PRPF8; a PSMD7; a pyroglutamyl-peptidase; or a combination thereof.

The protease may comprise a reelin; a renin; a retinol binding protein 3; a rhomboid 5 homolog; a rhomboid domain containing protease; or a combination thereof.

The protease may comprise a serine protease; a SADI; a secemin; a SUMO protease; a separase; a serine carboxypeptidase 1; a signalase 18 kDa component; a signalase 21 kDa component; a signalase-like 1 protease; a similar to Arabidopsis Ser-protease; a similar to SPUVE protease; a site-1 protease; a sonic hedgehog protein; a spinesin; a SprT-like N-terminal domain; a stromelysin; a suppressor of Ty 16 homolog; or a combination thereof.

The protease may comprise a taspase; a TBP-associated factor 2; a TESP2; a TESP3; a testase 2; a testis serine protease; a testisin; a testis-specific protein tsp50; a thimet oligopeptidase; a thrombin; a thymus-specific serine peptidase; a TINAG related protein; a TMPRSS11 A; a t-plasminogen activator; a TRAF-binding protein domain; a transferrin receptor protein; a transmembrane Ser-protease; a transthyretin; a TRH-degrading ectoenzyme; a tripeptidyl-peptidase; a trypsin; a tryptase; a tryptase homolog; a tubulointerstitial nephritis antigen; or a combination thereof.

The protease may comprise a ubiquitin C-term. hydrolase; a ubiquitin specific peptidase like 1; a UCR1; a UCR2; a UDP-N-acetylglucosaminyltransferase subunit; a Ufm-1 specific protease; a urokinase; an umbilical vein proteinase; a u-plasminogen activator; a USP; or a combination thereof.

The protease may comprise a VCP (p97)/p47-interacting protein; a VDU1; a vitellogenic carboxypeptidase-L; an X-Pro dipeptidase; a X-prolyl aminopeptidase 2; a YMEI-like 1; a zinc finger CCCH-type containing protease; a zinc finger containing ubiquitin peptidase 1; or a combination thereof.

The protease may comprise a cathepsin. Examples of suitable cathepsins include, but are not limited to, a cathepsin A (CTSA), a cathepsin B (CTSB), a cathepsin C (CTSC), a cathepsin D (CTSD), a cathepsin E (CTSE), a cathepsin H (CTSH), a cathepsin K (CTSK), a cathepsin L (CTSL), a cathepsin S (CTSS), a cathepsin V (CTSV), and a cathepsin Z (CTSZ). Cathepsins are a subset of proteases, many of which become activated in low pH. Cathepsins comprise serine proteases, cysteine proteases, and aspartyl proteases, among others. Cathepsins have been implicated in cancer, Alzheimer's disease, arthritis, Ebola, pancreatitis, glaucoma, COPD, and other diseases.

The protease may comprise a disintegrin and metalloproteinase (ADAM). ADAMs are a family of single-pass transmembrane and secreted metalloendopeptidases. Not all human ADAMs have a functional protease domain. Those ADAMs which are active proteases are classified as sheddases because they cut off or shed extracellular portions of transmembrane proteins. The ADAM may be ADAM-12.

The protease may comprise a furin. Furin belongs to the subtilisin-like proprotein convertase family, and is a calcium-dependent serine endoprotease. Furin's substrates include: proparathyroid hormone, transforming growth factor beta 1 precursor, proalbumin, pro-beta-secretase, membrane type-1 matrix metalloproteinase, beta subunit of pro-nerve growth factor and von Willebrand factor.

The protease may comprise a matrix metalloproteinase (MMP). MMPs are calcium-dependent zinc-containing endopeptidases. MMPs have been implicated in cleavage of cell surface receptors, the release of apoptotic ligands, chemokine/cytokine inactivation, cell proliferation and cell migration. The MMP may be MMP-2.

Suitable proteases include, but are not limited to, tumour-associated proteases and/or proteases associated with liver disease.

Examples of tumour-associated proteases include, but are not limited to, cathepsin B, C and D3 and a plasmin protease.

Examples of proteases associated with liver disease include, but are not limited to, FAP, MMP2, ADAMTS2, furin, MMP14, GZMB, PRSS8, MMP8, ADAM12, CTSS, CTSA, CTSZ, CASP1, ADAMTS12, CTSD, CTSW, MMP11, MMP12, GZMA, MMP23B, MMP7, ST14, MMP9, MMP15, ADAMDEC1, ADAMTS1, GZMK, KLK11, MMP19, PAPPA, CTSE, PCSK5 and/or PLAU.

The protease associated with liver disease may be selected from the group consisting of: fibroblast activation protein (FAP), a matrix metalloproteinase (MMP2), such as MMP-2, furin and a disintegrin and metalloproteinase (ADAM), such as ADAM-12.

Specific examples of glucuronidases that can be released from the lysosome into the extracellular space include β-glucuronidase and/or or β-galactosidase. In particular, the presence of β-glucuronidase in the extracellular space is a hall mark of cancer. Cancer-specific enzymes may be selected from β-glucuronidase, β-galactosidase α-L-arabinofuranosidase, N-acetyl-β-D-galactosaminidase, N-acetyl-β-D-glucosaminidase, hexosaminidase, α-L-fucosidase, α-galactosidase, α-glucosidase, β-glucosidase, α-L-iduronidase, α-mannosidase, β-mannosidase, lipases, phosphatases and/or sulfatases.

The step of detecting the presence of absence of the reporter molecule in the exhaled breath of the subject may be performed by any suitable method. For example, the exhaled breath sample may be captured and analysed according to the method, and using the device, as defined herein in relation to the kit of the present invention.

The step of detecting the presence of absence of the reporter molecule in the exhaled breath of the subject may be performed on site, for example by a suitable practitioner, or may be performed “off site”, i.e., the sample may be sent away for analysis. For example, an exhaled breath sample may be collected and fed into tubes containing a sorbent material, to which the compounds of interest adsorb. After sufficient sample has been obtained, the sorbent tubes may be removed from the device and sent away for analysis.

The concentration or amount of the reporter molecule may be determined in absolute or relative terms in multiple biological matrix samples. For example, where the concentration is determined in an exhaled breath sample, it may be determined in a first breath sample (collected at a first time period) and in a second breath sample (collected at a later, second time period), thus permitting analysis of the kinetics or rate of change of concentration thereof over time.

A sample of exhaled breath may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of the reporter molecule in a sample of exhaled breath from a subject may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Isotope ratio mass spectrometry (IRMS), Electronic Nose device, quartz crystal microbalance or chemically sensitive sensors.

The amount of the reporter molecule in a sample of exhaled breath from a subject may be determined using thermal desorption-gas chromatography-time of flight-mass spectrometry (GC-Tof-MS). For example, in one embodiment the breath of the subject is collected in an inert bag, then the content of the bag is transported under standardised conditions onto desorption tubes and VOCs are analyzed by thermally desorbing the content of the tube and then separated by capillary gas chromatography. Then volatile organic peaks are detected with MS and identified using for example a library, such as the National Institute of Standards and Technology. Thermal desorption may be performed at the GC inlet at a temperature of, e.g., about 200-350° C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine one or more VOCs from a sample of exhaled breath from a subject.

The method may comprise collecting different selected exhaled breath samples, or fractions thereof, on a single breath sample capture device, the method comprising the steps of:
(a) collecting a first exhaled breath sample by contacting the sample with a capture device comprising an adsorbent material;
(b) collecting a second exhaled breath sample by contacting the second sample with said capture device, wherein the first and second exhaled breath samples are caused to be captured on the capture device in a spatially separated manner.

The capture device may comprise an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. The material may be Tenax GR, which comprises a mixture of Tenax® TA and 30% graphite. One Carbograph® adsorbent is Carbograph 5TD. The capture device may comprise both Tenax GR and Carbograph 5TD. The capture device may conveniently be a sorbent tube. These are hollow metal cylinders, typically of standard dimensions (3½ inches in length with a ¼ inch internal diameter) packed with a suitable adsorbent material.

When an appropriate reference is indicative of a subject being free of a disease, a detectable difference (e.g., a statistically significant difference) between the value determined from a subject in need of characterization or diagnosis of a disease and the appropriate reference may be indicative of the disease in the subject. When an appropriate reference is indicative of the disease, a lack of a detectable difference (e.g., lack of a statistically significant difference) between the value determined from a subject in need of characterization or diagnosis of a disease and the appropriate reference may be indicative of the disease in the subject.

Thus, the methods may include detecting the concentration of the reporter molecule in a biological matrix sample from the subject, and diagnosing the subject as having a likelihood of a disease state if the level of one or more of the substrate and/or metabolite is different from the healthy subject value.

The methods of the invention may further include the step of selecting a treatment for said disease. The methods may further include administering said treatment to said subject.

### Use

The invention also relates to the use of a compound and/or composition and/or kit according to the invention in a method of the invention.

The invention also relates to the use of a compound and/or composition and/or kit according to the invention in a method of detecting or monitoring a disease.

### Selected Definitions

The term “alk” or “alkyl”, as used herein unless otherwise defined, relates to saturated hydrocarbon radicals being straight, branched, cyclic or polycyclic moieties or combinations thereof and contain 1 to 20 carbon atoms, such as 1 to 10 carbon atoms, such as 1 to 8 carbon atoms, such as 1 to 6 carbon atoms, or even 1 to 4 carbon atoms. These radicals may be optionally substituted with a chloro, bromo, iodo, cyano, nitro, OR¹⁹, OC(O)R²⁰, C(O)R²¹, C(O)OR²², NR²³R²⁴, C(O)NR²⁵R²⁶, SR²⁷, C(O)SR²⁷, C(S)NR²⁵R²⁶, aryl or Het, wherein R¹⁹ to R²⁷ each independently represent hydrogen, aryl or alkyl, and/or be interrupted by oxygen or sulphur atoms, or by silano or dialkylsiloxane groups. Examples of such radicals may be independently selected from methyl, ethyl, n-propyl, isopropyl, n-butyl, isobutyl, sec-butyl, tert-butyl, 2-methylbutyl, pentyl, iso-amyl, hexyl, cyclohexyl, 3-methylpentyl, octyl and the like. The term “alkylene”, as used herein, relates to a bivalent radical alkyl group as defined above. For example, an alkyl group such as methyl which would be represented as —CH₃, becomes methylene, —CH₂—, when represented as an alkylene. Other alkylene groups should be understood accordingly.

The term “alkenyl”, as used herein, relates to hydrocarbon radicals having, such as up to 4, double bonds, being straight, branched, cyclic or polycyclic moieties or combinations thereof and containing from 2 to 18 carbon atoms, such as 2 to 10 carbon atoms, such as from 2 to 8 carbon atoms, such as 2 to 6 carbon atoms, or even 2 to 4 carbon atoms. These radicals may be optionally substituted with a hydroxyl, chloro, bromo, iodo, cyano, nitro, OR¹⁹, OC(O)R²⁰, C(O)R²¹, C(O)OR²², NR²³R²⁴, C(O)NR²⁵R²⁶, SR²⁷, C(O)SR²⁷, C(S)NR²⁵R²⁶, or aryl, wherein R¹⁹ to R²⁷ each independently represent hydrogen, aryl or alkyl, and/or be interrupted by oxygen or sulphur atoms, or by silano or dialkylsiloxane groups. Examples of such radicals may be independently selected from alkenyl groups include vinyl, allyl, isopropenyl, pentenyl, hexenyl, heptenyl, cyclopropenyl, cyclobutenyl, cyclopentenyl, cyclohexenyl, 1-propenyl, 2-butenyl, 2-methyl-2-butenyl, isoprenyl, farnesyl, geranyl, geranylgeranyl and the like. The term “alkenylene”, as used herein, relates to a bivalent radical alkenyl group as defined above. For example, an alkenyl group such as ethenyl which would be represented as —CH═CH₂, becomes ethenylene, —CH═CH—, when represented as an alkenylene. Other alkenylene groups should be understood accordingly.

The term “alkynyl”, as used herein, relates to hydrocarbon radicals having, such as up to 4, triple bonds, being straight, branched, cyclic or polycyclic moieties or combinations thereof and having from 2 to 18 carbon atoms, such as 2 to 10 carbon atoms, such as from 2 to 8 carbon atoms, such as from 2 to 6 carbon atoms, or even from 2 to 4 carbon atoms. These radicals may be optionally substituted with a hydroxy, chloro, bromo, iodo, cyano, nitro, OR¹⁹, OC(O)R²⁰, C(O)R²¹, C(O)OR²², NR²³R²⁴, C(O)NR²⁵R²⁶, SR²⁷, C(O)SR²⁷, C(S)NR²⁵R²⁶, or aryl, wherein R¹⁹ to R²⁷ each independently represent hydrogen, aryl or lower alkyl, and/or be interrupted by oxygen or sulphur atoms, or by silano or dialkylsiloxane groups. Examples of such radicals may be independently selected from alkynyl radicals include ethynyl, propynyl, propargyl, butynyl, pentynyl, hexynyl and the like. The term “alkynylene”, as used herein, relates to a bivalent radical alkynyl group as defined above. For example, an alkynyl group such as ethynyl which would be represented as —C≡CH, becomes ethynylene, —C≡C—, when represented as an alkynylene. Other alkynylene groups should be understood accordingly.

The term “aryl” as used herein, relates to an organic radical derived from an aromatic hydrocarbon by removal of one hydrogen, and includes any monocyclic, bicyclic or polycyclic carbon ring of up to 7 members in each ring, wherein a ring is aromatic. These radicals may be optionally substituted with a hydroxy, chloro, bromo, iodo, cyano, nitro, OR¹⁹, OC(O)R²⁰, C(O)R²¹, C(O)OR²², NR²³R²⁴, C(O)NR²⁵R²⁶, SR²⁷, C(O)SR²⁷, C(S)NR²⁵R²⁶, or aryl, wherein R¹⁹ to R²⁷ each independently represent hydrogen, aryl or lower alkyl, and/or be interrupted by oxygen or sulphur atoms, or by silano or dialkylsilcon groups. Examples of such radicals may be independently selected from phenyl, p-tolyl, 4-methoxyphenyl, 4-(tert-butoxy)phenyl, 3-methyl-4-methoxyphenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, tetrahydronaphthyl, indanyl, biphenyl, phenanthryl, anthryl or acenaphthyl and the like. The term “arylene”, as used herein, relates to a bivalent radical aryl group as defined above. For example, an aryl group such as phenyl which would be represented as -Ph, becomes phenylene, -Ph-, when represented as an arylene. Other arylene groups should be understood accordingly.

The term “cycloalkyl”, as used herein, refers to any of the above mentioned alkyl groups which comprise one or more cyclic group(s). The term “cycloalkylene”, as used herein, relates to a bivalent radical cycloalkyl group as defined above. For example, a cycloalkyl group such as cyclohexyl which would be represented as —C₆H₁₁, becomes cyclohexylene, —C₆H₁₀—, when represented as a cycloalkylene. Other cycloalkylene groups should be understood accordingly. It will be understood that the bivalent radical may be attached to two (organic) groups via any suitable atoms on the ring. For example, the groups may be attached on adjacent atoms or otherwise (for example, at the meta, ortho or para position, as appropriate).

The terms “het” or “hetero” refer to sulphur, phosphorus, oxygen and/or nitrogen atoms. As such, “heteroalkyl”, “heteroalkenyl” and “heteroakynyl” etc. groups are those that contain at least one sulphur, phosphorus, oxygen and/or nitrogen atom in the alkyl, alkenyl, alkynyl etc. chain. “Heterocyclic” groups are those that contain at least one sulphur, phosphorus, oxygen and/or nitrogen atom in an alkyl or aryl ring. The ring that includes the heteroatom can be aromatic or nonaromatic. Representative examples of heterocyclic groups include, but are not limited to, benzofuranyl, benzothiophene, indolyl, benzopyrazolyl, pyrrolyl, thiophenyl (thiopene), furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. The heterocyclic groups may be substituted with groups including, but not limited to, -alkyl, —O-(alkyl), aryl, —C(O)R′, —OC(O)R′, —C(O)OR′, —C(O)NH₂, —C(O)NHR′, —C(O)N(R′)2, —NHC(O)R′, —S(O)₂R′, —S(O)R′, —OH, -halogen, —N₃, —NH₂, —NH(R′), —N(R′)₂ and —CN; wherein alkyl is preferably C₁-C₈alkyl and each R′ is independently selected from —H, -alkyl, such as C₁-C₈ alkyl and aryl. A substituted heterocyclic group may also include one or more of: —NHC═NH) NH₂, —NHCONH₂, —S(O)₂R′ and —SR′.

For the avoidance of doubt, the reference to alkyl, alkenyl, alkynyl, aryl or aralkyl in composite groups herein should be interpreted accordingly, for example the reference to alkyl in aminoalkyl or alk in alkoxyl should be interpreted as alk or alkyl above etc.

As used herein, unless otherwise expressly specified, all numbers such as those expressing values, ranges, amounts or percentages may be read as if prefaced by the word “about”, even if the term does not expressly appear. Also, the recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

The terms “comprising”, “comprises” and “comprised of” as used herein are synonymous with “including”, “includes” or “containing”, “contains”, and are inclusive or open-ended and do not exclude additional, non-recited members, elements or method steps.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

### EXAMPLES

Exemplary compounds according to the invention are as follows:

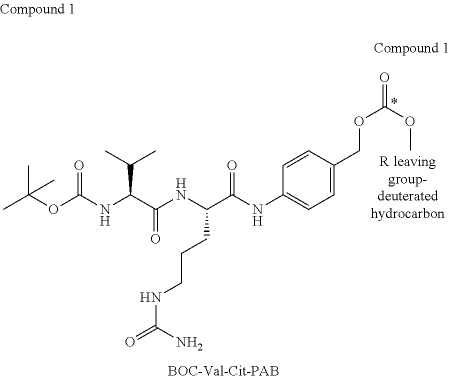 [SMILES: COC(=O)OCc1ccc(NC(=O)[C@H](CCCNC(N)=O)NC(=O)[C@@H](NC(=O)OC(C)(C)C)C(C)C)cc1]

Compound 1 is as shown above, wherein m is 0; the cleavable group, Y, is tert-butyloxycarbonyl (BOC)-Val-Cit; the self-immolative linker, L, is para-aminobenzyl carbonate isotopically labelled at the carbonyl carbon of the carbonate group; and the terminating moiety, T, is deuterated ethyl. Upon cleavage of the Val-Cit peptide by Cathepsin B in vivo, and self-immolation of the self-immolative linker group, isotopically labelled carbon dioxide (CO₂) and D5-ethanol are released as reporter molecules.

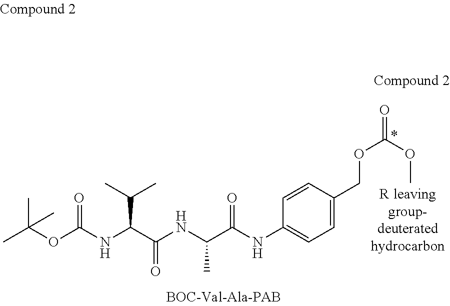 [SMILES: COC(=O)OCc1ccc(NC(=O)[C@H](C)NC(=O)[C@@H](NC(=O)OC(C)(C)C)C(C)C)cc1]

Compound 2 is as shown above, wherein m is 0; the cleavable group, Y, is tert-butyloxycarbonyl (BOC)-Val-Ala; the self-immolative linker, L, is para-aminobenzyl carbonate isotopically labelled at the carbonyl carbon of the carbonate group; and the terminating moiety, T, is deuterated ethyl. Upon cleavage of the Val-Cit peptide by Cathepsin B in vivo, and self-immolation of the self-immolative linker group, isotopically labelled carbon dioxide (CO2) and D5 ethanol are released as reporter molecules.

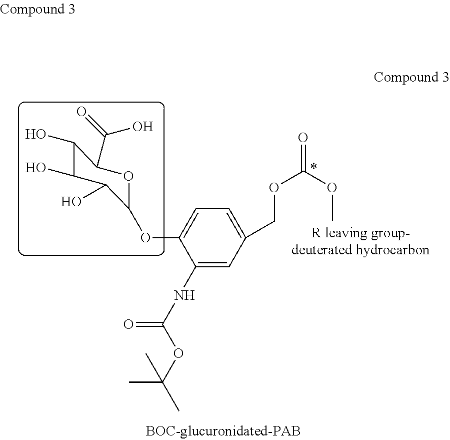 [SMILES: COC(=O)OCc1ccc(OC2OC(C(=O)O)C(O)C(O)C2O)c(NC(=O)OC(C)(C)C)c1]

Compound 3 is as shown above, wherein m is 0; the cleavable group, Y, is a glucuronide group which is covalently attached to the aromatic ring of the self-immolative linker, L, at the para-position relative to the methylene carbonate group; the self-immolative linker, L, is meta-aminobenzyl carbonate isotopically labelled at the carbonyl carbon of the carbonate group; and the terminating moiety, T, is deuterated ethyl. Upon cleavage of the glucuronide group by β-glucuronidase in vivo, and self-immolation of the self-immolative linker group, L, isotopically labelled carbon dioxide (CO2) and D5 ethanol are released as reporter molecules.

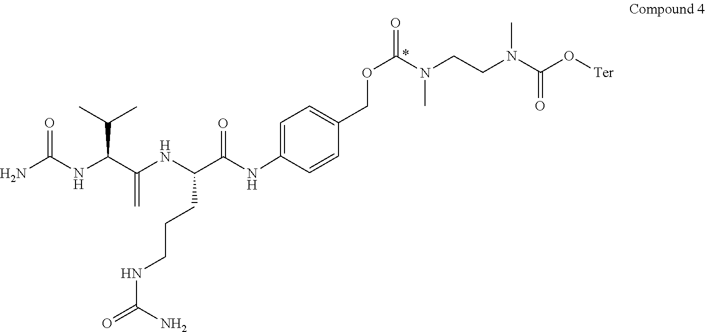

Compound 4 is as shown above, wherein m is 0; the cleavable group, Y, is Val-Cit; the self-immolative linker, L, is para-aminobenzyl carbamate isotopically labelled at the carbonyl carbon of the carbamate group which also comprises a self-cyclizing spacer group of the formula —CH₂—CH₂—N(CH₃)—C(O)—O—; and the terminating moiety, T, is Ter, which represents deuterated ethyl. Upon cleavage of the Val-Cit peptide by Cathepsin B in vivo, and self-immolation of the self-immolative linker group, isotopically labelled carbon dioxide (CO2) and D5 ethanol are released as reporter molecules. Iminoquinone methide and 1,3-dimethyl-2-imidazole are also released.

The released isotopically labelled carbon dioxide (CO2) and D5 ethanol from each of compounds 1-4 may be detected in breath samples obtained from a subject to detect or prognose a cancer by the following method. The compounds (1-4) may be administered intravenously to a subject suspected of having a cancer. If the subject has a cancer, the compounds will be cleaved in vivo by Cathepsin B or β-glucuronidase in the microenvironment of the tumour, releasing the reporter molecules described above.

An exhaled breath sample may then be taken from the subject using the device as described in WO2017/187141 (which is fully incorporated herein). The exhaled breath samples are fed into tubes containing a sorbent material, to which the compounds of interest, i.e., reporter molecules, adsorb. After sufficient sample has been obtained, the sorbent tubes are removed from the sampling device and the adsorbed compounds desorbed (by heating) and subjected to analysis by FAIMS to identify the presence and/or amount of the reporter molecules described above. If the reporter molecules are present in the exhaled breath sample from the patient, it is indicative that the subject has a cancer.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

Whereas particular examples of this invention have been described above for purposes of illustration, it will be evident to those skilled in the art that numerous variations of the details of the present invention may be made without departing from the invention as defined in the appended claims.

1. A compound of the formula Xₘ—(Y-L-T)ₙ, wherein X, when present is a moiety operable to target the compound to a site of interest in vivo; m is 0 or at least 1; Y is a cleavable group; L is a self-immolative linker; T is a terminating moiety; and n is 1 when m is 0 or n is at least 1 when m is at least 1; wherein at least one of the self-immolative linker, L, and terminating moiety, T, comprise an isotopically labelled reporter molecule or a precursor thereof; wherein the reporter molecule is operable to be released upon cleavage of the cleavable group, Y; and wherein the reporter molecule, upon release, is a volatile compound.

2. The compound according to claim 1, wherein m is at least 1 and n is at least 1.

3. The compound according to any preceding claim, wherein m is 1 and n is 1.

4. The compound according to any preceding claim, wherein X comprises an antigen-binding moiety, a ligand for a cell surface receptor, a pHLIP, and/or a nanoparticle.

5. The compound according to claim 4, wherein X is an antigen-binding moiety.

6. The compound according to claim 5, wherein the antigen-binding moiety comprises an antibody and/or an antibody mimetic, for example a cyclic peptide, a cysteine knot and/or an anticalin.

7. The compound according to claim 6, wherein X is cyclic peptide, for example a bicyclic peptide.

8. The compound according to claim 7, wherein X is a peptide comprising at least three cysteine residues, separated by at least two loop sequences, and a molecular scaffold which forms covalent bonds with the cysteine residues of the polypeptide such that at least two polypeptide loops are formed on the molecular scaffold, wherein the peptide ligand comprises an amino acid sequence of formula:
-C-X₁-U/O-X₃-X₄-G-C-E-D-F-Y-X₁₀-X₁₁-C- (SEQ ID NO: 1)
or a pharmaceutically acceptable salt thereof; wherein X represents any amino acid residue; U represents a polar, uncharged amino acid residue selected from the group consisting of N, C, Q, M, S and T; and O represents a non-polar aliphatic amino acid residue selected from the group consisting of G, A, I, L, P and V.

9. The compound according to claim 8, wherein X₁ is selected from Y, M, F or V, for example Y.

10. The compound according to any of claim 8 or 9, wherein U/O is a U, for example N.

11. The compound according to any of claim 8 or 9, wherein U/O is an O, for example G.

12. The compound according to any of claims 8-11, wherein X₃ is selected from N, C, Q, M, S, T, D or E, for example Q or E.

13. The compound according to any of claims 8-12, wherein X₄ is selected from F, W or Y.

14. The compound according to any of claims 8-13, wherein X₁₀ is selected from D or E, for example D.

15. The compound according to any of claims 8-14, wherein X₁₁ is selected from G, A, I, L, P or V, for example I.

16. The compound according to claim 4, wherein X comprises a ligand for a cell-surface receptor.

17. The compound according to claim 16, wherein the ligand comprises folic acid, folate, and/or a peptide hormone.

18. The compound according to claim 17, wherein the peptide hormone comprises a somatostatin and/or a somatostatin analogue.

19. The compound according to any preceding claim, wherein the self-immolative linker, L, comprises a methylene carbamate group, an aminobenzyl carbamate group, an aminobenzyl carbonate group, an aminobenzyl ether group, a dithiobenzyl carbamate group, a dithiobenzyl carbonate group and/or a dithiobenzyl ether group.

20. The compound according to claim 19, wherein the self-immolative linker, L, comprises an aminobenzyl carbamate group and/or an aminobenzyl carbonate group, for example a p-aminobenzyl carbamate group and/or a p-aminobenzyl carbonate group.

21. The compound according to any of claim 19 or 20, wherein the self-immolative linker comprises a spacer group between the terminating moiety, T, and the methylene carbamate group, aminobenzyl carbamate group, aminobenzyl carbonate group, aminobenzyl ether group, dithiobenzyl carbamate group, dithiobenzyl carbonate group and/or dithiobenzyl ether group.

22. The compound according to claim 21, wherein the spacer group comprises a self-cyclizing group, for example of the formula —(CH₂)ₙ—NR—C(O)—O—, wherein n is 2 to 10 and each R is independently hydrogen or a C1-C6 alkyl group.

23. The compound according to claim 20, wherein n is 2 and each R is methyl.

24. The compound according to any preceding claim, wherein the self-immolative linker is of the formula: 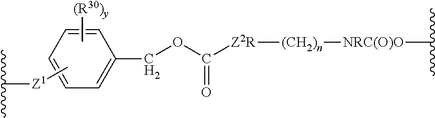
wherein y is an integer from 0 to 4; R³⁰ is a substituent on the aromatic ring which, when present, replaces a hydrogen that is otherwise attached to one of the four non-substituted carbons that form the ring; Z¹ is —NH— or —S—S—; Z² is N or O; n is 2 to 10; and each R is independently hydrogen or a C1-C6 alkyl group.

25. The compound according to claim 24, wherein Z is O, n is 2 and each R is methyl.

26. The compound according to any preceding claim, wherein the terminating moiety, T, comprises hydrogen, a C1-C10 alkyl group, a C2-C10 alkenyl group, a C2-C10 alkynyl group, an aryl group, a therapeutically active agent and/or a molecular label.

27. The compound according to claim 26, wherein the terminating moiety, T, comprises a C1-C10 alkyl group and/or a C2-C10 alkenyl group, such as an ethyl group and/or a hexenyl group.

28. The compound according to any preceding claim, wherein the self-immolative linker, L, comprises a reporter molecule.

29. The compound according to claim 28, wherein the reporter molecule comprises isotopically labelled carbon dioxide and/or an isotopically labelled heterocyclic compound.

30. The compound according to claim 29, wherein the reporter molecule comprises isotopically labelled carbon dioxide.

31. The compound according to any preceding claim, wherein the terminating moiety, T, comprises a reporter molecule, for example a C1-C10 alkanol and/or a C2-C10 alkenol, or for example ethanol and/or trans-2-hexenol.

32. The compound according to any preceding claim, wherein the reporter molecule comprises carbon dioxide, a C1-C10 alkanol, a C2-C10 alkenol, a C1-C10 alkylamine, 2-imidazolidinone, a 1,3-di(C1-C6 alkyl)-2-imidazolidonone, iminoquinone methide and/or aminobenzyl alcohol.

33. The compound according to claim 32, wherein the reporter molecule comprises carbon dioxide, a C1-C10 alkanol and/or a C2-C10 alkenol, for example carbon dioxide, ethanol and/or trans-2-hexenol.

34. The compound according to any preceding claim, wherein the isotopic label is 12C, 13C, 14C, 2H, 17O and/or 18O.

35. The compound according to any preceding claim, wherein the reporter molecule comprises at least two different isotopic labels.

36. The compound according to any preceding claim, wherein the cleavable group, Y, comprises a peptide and/or a glucuronide group.

37. The compound according to claim 36, wherein the peptide comprises a dipeptide, for example a dipeptide selected from the group consisting of Val-Ala, Ala-Ala and Val-Cit.

38. The compound according to claim 36, wherein the glucuronide unit comprises a sugar moiety, Su, comprising a substituted β-D-glucuronide, for example ethyl-βD-glucuronide, or for example D5-ethyl-βD-glucuronide.

39. The compound according to any preceding claim, comprising a further cleavable group, Y′, for example a further cleavable group covalently attached to the self-immolative group, L, such that the compound is of the formula Xₘ—(Y-L(Y′)-T)ₙ.

40. The compound according to any of claim 1 or 19-39, wherein m is 0 and n is 1.

41. The compound according to any preceding claim, wherein the cleavable group, Y, comprises a peptide and/or a glucuronide group; the self-immolative linker, L, comprises an isotopically labelled aminobenzyl carbamate or aminobenzyl carbonate group; and the reporter molecule comprises isotopically labelled carbon dioxide.

42. The compound according to any of claims 1-40, wherein cleavable group, Y, comprises a peptide and/or a glucuronide group; the self-immolative linker, L, comprises an aminobenzyl carbonate group; the terminating moiety, T, comprises an isotopically labelled C1-C10 alkyl and/or C1-C10 alkenyl group; and the reporter molecule comprises an isotopically labelled C1-C10 alkanol and/or C1-C10 alkenol.

43. The compound according to any of claims 1-40, wherein the cleavable group, Y, comprises a peptide and/or a glucuronide group; the self-immolative linker, L, comprises an isotopically labelled aminobenzyl carbonate group; the terminating moiety, T, comprises an isotopically labelled C1-C10 alkyl and/or C1-C10 alkenyl group; and the reporter molecule comprises isotopically labelled carbon dioxide and an isotopically labelled C1-C10 alkanol and/or C1-C10 alkenol.

44. The compound according to any of claims 1-40, wherein the cleavable group, Y, comprises a peptide and/or a glucuronide group; the self-immolative linker, L, is of the formula: 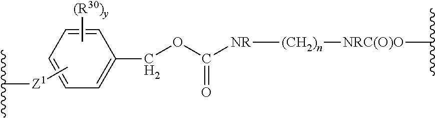
wherein y is an integer from 0 to 4; R³⁰ is a substituent on the aromatic ring which, when present, replaces a hydrogen that is otherwise attached to one of the four non-substituted carbons that form the ring; Z¹ is —NH— or —S—S—; each R is independently hydrogen or a C1-C6 alkyl group; the terminating moiety, T, comprises a C1-C10 alkyl and/or C1-C10 alkenyl group; and the reporter molecule comprises carbon dioxide, 2-imidazolidinone, 1,3-di(C1-C6 alkyl)-2-imidazolidonone, a C1-C10 alkanol and/or a C1-C10 alkenol.

45. A composition comprising one or more compound(s) according to any one of claims 1-44.

46. A method for the detection or prognosis of a disease comprising administering a compound according to any one of claims 1-44 or a composition according to claim 45 to a subject and detecting the presence or absence of the released reporter molecule in the exhaled breath of the subject.

47. The method according to claim 46, wherein the method comprises establishing a test subject value based on a concentration of the released reporter molecule.

48. The method according to claim 47, wherein the test subject value is compared to one or more reference value(s) and wherein a difference in the test subject value and a reference value indicates a likelihood of disease.

49. The method according to claim 48, wherein the reference value is the value of a subject that has been diagnosed with a disease of interest.

50. The method according to claim 48, wherein the reference value is the value of a healthy subject and/or is the value of the subject prior to administration of the compound according to any one of claims 1-44 and/or the composition according to claim 45.

51. The method according to any one of claims 46-50, wherein the method comprises collection of a breath sample from said subject.

52. The method according to any one of claims 46-51, wherein the disease is a liver disease, such as non-alcoholic steatohepatitis (NASH), non-alcoholic fatty liver disease (NAFLD) or nonalcoholic steatohepatitis (HCC), or a cancer.

53. A kit comprising the compound according to any one of claims 1-44 and/or the composition according to claim 45 and a device for capturing a breath sample from a subject.

54. Use of a compound according to any one of claims 1-44, composition according to claim 45 and/or kit according to claim 53 in a method according to any one of claims 46-52.

55. Use of a compound according to any one of claims 1-44, composition according to claim 45 and/or kit according to claim 53 in a method of detecting or monitoring a disease.

56. The use according to claim 55, wherein the disease is a cancer.

57. The method according to claim 52 or use according to claim 56, wherein the cancer is selected from the group consisting of: lung cancer, breast cancer, ovarian cancer, bowel cancer, prostate cancer, bladder cancer, colorectal cancer, pancreas carcinoma, kidney cancer, renal cancer, brain cancer and other CNS and intracranial tumours cancer, head and neck cancer, oesophageal cancers, sarcoma and carcinomas, mesothelioma, osteosarcoma, endometrial cancer and melanoma.


SEQUENCE LISTING (20 sequences)

SEQ ID NO: 1 - protein, 14 aa, synthetic construct
  VARIANT 3: note = N, C, Q, M, S, T, G, A, I, L, P or V
CXXXXGCEDFYXXC

SEQ ID NO: 2 - protein, 14 aa, synthetic construct
  VARIANT 3: note = N, C, Q, M, S or T
CXXXXGCEDFYXXC

SEQ ID NO: 3 - protein, 14 aa, synthetic construct
  VARIANT 3: note = G, A, I, L, P or V
CXXXXGCEDFYXXC

SEQ ID NO: 4 - protein, 28 aa, synthetic construct
ALDDQNPWRAYLDLLFPTDTLLLDLLWA

SEQ ID NO: 5 - protein, 28 aa, synthetic construct
ACDDQNPWRAYLDLLFPTDTLLLDLLWA

SEQ ID NO: 6 - protein, 28 aa, synthetic construct
  VARIANT 2: note = X can be any amino acid
AXDDQNPWRAYLDLLFPTDTLLLDLLWA

SEQ ID NO: 7 - protein, 28 aa, synthetic construct
ALDQDNPWRAYLDLLFPTDTLLLDLLWA

SEQ ID NO: 8 - protein, 28 aa, synthetic construct
ACDQDNPWRAYLDLLFPTDTLLLDLLWA

SEQ ID NO: 9 - protein, 28 aa, synthetic construct
  NON_STD 2: note = azido-containing amino acid
AXDQDNPWRAYLDLLFPTDTLLLDLLWA

SEQ ID NO: 10 - protein, 26 aa, synthetic construct
  VARIANT 2..4: note = X can be any amino acid
AXXXPWRAYLDLLFPTDTLLLDLLWA

SEQ ID NO: 11 - protein, 27 aa, synthetic construct
  VARIANT 2..5: note = X can be any amino acid
AXXXXPWRAYLDLLFPTDTLLLDLLWA

SEQ ID NO: 12 - protein, 28 aa, synthetic construct
  VARIANT 2..6: note = X can be any amino acid
AXXXXXPWRAYLDLLFPTDTLLLDLLWA

SEQ ID NO: 13 - protein, 29 aa, synthetic construct
  VARIANT 2..7: note = X can be any amino acid
AXXXXXXPWRAYLDLLFPTDTLLLDLLWA

SEQ ID NO: 14 - protein, 30 aa, synthetic construct
  VARIANT 2..8: note = X can be any amino acid
AXXXXXXXPWRAYLDLLFPTDTLLLDLLWA

SEQ ID NO: 15 - protein, 31 aa, synthetic construct
  VARIANT 2..9: note = X can be any amino acid
AXXXXXXXXPWRAYLDLLFPTDTLLLDLLWA

SEQ ID NO: 16 - protein, 32 aa, synthetic construct
  VARIANT 2..10: note = X can be any amino acid
AXXXXXXXXXPWRAYLDLLFPTDTLLLDLLWA

SEQ ID NO: 17 - protein, 33 aa, synthetic construct
  VARIANT 2..11: note = X can be any amino acid
AXXXXXXXXXXPWRAYLDLLFPTDTLLLDLLWA

SEQ ID NO: 18 - protein, 34 aa, synthetic construct
  VARIANT 2..12: note = X can be any amino acid
AXXXXXXXXXXXPWRAYLDLLFPTDTLLLDLLWA

SEQ ID NO: 19 - protein, 35 aa, synthetic construct
  VARIANT 2..13: note = X can be any amino acid
AXXXXXXXXXXXXPWRAYLDLLFPTDTLLLDLLWA

SEQ ID NO: 20 - protein, 31 aa, synthetic construct
  VARIANT 2: note = X can be any amino acid
AXQDNDQNXPWRAYLDLLFPTDTLLLDLLWA